SEQUENCE LISTING (10 sequences)

SEQ ID NO: 1 - DNA, 1281 bp, Artificial Sequence
  IgER1-Fc receptor fusion
atggttccacaaaaaccaaaagttagtcttaatccaccttggaatcgtatttttaaaggtgaaaatgttacacttacttgtaatggtaataatttttttgaagtatcttctacaaaatggtttcacaatggttcactttcagaagaaactaattcatcacttaatattgttaatgcaaaatttgaagattcaggtgaatataaatgtcaacatcaacaagttaatgaatctgaacctgtttatcttgaagtttttagtgattggttacttttacaagcatcagcagaagttgttatggaaggacaaccactttttcttcgttgtcatggttggagaaattgggatgtatataaagttatttattataaagatggtgaagctttaaaatattggtatgaaaatcataatatttcaattacaaatgctactgttgaagatagtggtacttattattgtacaggtaaagtttggcaacttgattatgaatcagaacctcttaatattactgttattaaagaaccaaaatcttgtgataaaacacacacttgtcctccatgtcctgctcctgaacttttaggaggaccatcagtatttcttttcccaccaaaacctaaagatacacttatgatttcacgtacacctgaagttacttgtgtagtagtagatgtttcacatgaagatccagaagttaaatttaattggtatgttgatggagtagaagttcacaatgcaaaaactaaaccacgtgaagaacaatataattctacatatcgtgtagtttcagttcttactgttcttcaccaagattggcttaatggtaaagaatataaatgtaaagtatcaaataaagcacttcctgcaccaattgaaaaaactatttcaaaagcaaaaggacaacctcgtgaaccacaagtttatacacttccaccatctcgtgatgaacttacaaaaaatcaagtatcacttacttgccttgtaaaaggtttctatccatcagatattgctgttgaatgggaatcaaatggtcaaccagaaaataattataaaacaactcctccagttttagattcagatggatcattcttcctttattcaaaacttacagtagataaatctcgttggcaacaaggaaatgttttctcatgttctgtaatgcacgaagctcttcacaatcactatacacaaaaatctctttctctttcacctggtaaagactataaagatcacgatggtgattataaagaacacgatattgattacaaagatgatgatgataaataa

SEQ ID NO: 2 - protein, 426 aa, Artificial Sequence
  IgER1-Fc receptor fusion
MVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDYKDHDGDYKEHDIDYKDDDDK

SEQ ID NO: 3 - DNA, 729 bp, Mouse
atggattacaaagaccatgatggtgactacaaagatcatgacattgattataaagatgatgatgataaagatgttgttatgacacaaacaccattatcattaccagtttcattaggtgatcaagcatcaatttcatgtcgttcatcacaatcattagttcattcaaatggtaatacatatttacattggtatttacaaaaaccaggtcaatcaccaaaattattaatttataaagtttcaaatcgtttttctggtgtaccagatcgtttttctggttctggttctggtacagattttacattaaaaatttctcgtgtagaagctgaagatttaggtgtatatttttgttctcaatctacacatgtaccttggactttaggtggtggtaccaaattagaaattaaacgtgctgacgctgctcctactgtatctatttttcctcctagttcagaacaattaacaagtggtggtgctagtgttgtttgttttttaaataatttctatccaaaagatattaatgttaaatggaaaattgatggttcagaacgtcaaaatggtgttttaaattcatggacagatcaagattcaaaagattcaacatactcaatgtcatcaacattaacattaactaaagatgaatacgaacgtcataattcatacacttgtgaagcaactcataaaacttcaacatcaccaattgttaaatcatttaatcgtaatgaatgttaa

SEQ ID NO: 4 - protein, 242 aa, Mouse
  MISC_FEATURE (1)..(23): FLAG tag
MDYKDHDGDYKDHDIDYKDDDDKDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTLGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 1380 bp, Mouse
atggaagttaaattagttgaatcaggtggtggtttagttaaaccaggaggttcattaaaattatcttgtgctgcttctggttttacattttctagttatgctatgtcatgggttcgtcaaactcctgaaaaacgtttagaatgggtagcatcaatttcatcaggtggttcaacttattatcctgattctgtaaaaggtcgtttcactattttccgtgataatgcacgtaatattttatacttacaaatgtcttctttacgttcagaagatacagctatgtactattgtgtacgtggtcaagactattggggtcaaggtacatcagtaacagtatcatcagctaaaacaacaccaccatcagtttatccattagctccaggttctgctgctcaaactaattctatggttactttaggttgtttagttaaaggttattttcctgaacctgtaactgtaacttggaatagtggttctttatcttctggtgtacatacttttcctgctgtattacaatctgatttatatactttatcttcttcagtaacagtaccatcatcaacatggccatcagaaacagtaacatgtaatgtagcacatccagcatcatcaacaaaagtagataaaaaaattgttccaagagattgtggttgtaaaccatgtatttgtacagttccagaagtttcatcagtttttattttcccaccaaaacctaaagatgttttaacaattacattaacacctaaagttacatgtgttgtagtagatatttcaaaagatgatcctgaagttcaattctcatggtttgttgatgatgttgaagttcacacagcacaaacacaaccaagagaagaacaatttaattcaacatttagatcagtttcagaattaccaattatgcatcaagattggttaaatggtaaagaatttaaatgtagagttaattcagcagcattcccagcaccaattgaaaaaacaatttcaaaaacaaaaggaagaccaaaagctccacaagtttatacaattccacctccaaaagaacaaatggctaaagataaagtttcattaacatgtatgattacagatttcttcccagaagatattacagttgaatggcaatggaatggtcaaccagctgaaaattataaaaatactcaacctattatggatactgatggttcatattttgtttattcaaaattaaatgttcaaaaatctaattgggaagctggtaatacttttacatgttctgtattacatgaaggtttacacaatcatcatacagaaaaaattttatctcattctccaggtaaagactataaagatcacgatggtgattataaagaacacgatattgattacaaagatgatgatgataaataa

SEQ ID NO: 6 - protein, 459 aa, Mouse
  MISC_FEATURE (438)..(459): FLAG tag
MEVKLVESGGGLVKPGGSLKLSCAASGFTFSSYAMSWVRQTPEKRLEWVASISSGGSTYYPDSVKGRFTIFRDNARNILYLQMSSLRSEDTAMYYCVRGQDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKILSHSPGKDYKDHDGDYKEHDIDYKDDDDK

SEQ ID NO: 7 - DNA, 735 bp, Artificial Sequence
  Chimeric human kappa light chain
atggattacaaagaccatgatggtgactacaaagatcatgacattgattataaagatgatgatgataaagatgtagtaatgacacaaacaccattatcattaccagtttcattaggtgatcaagcttcaatttcatgtcgttcaagtcaaagtttagttcatagtaatggtaatacttatttacattggtatttacaaaaaccaggtcaatctcctaaattattaatttataaagtttctaaccgtttttctggtgttcctgaccgtttttctggatctggttctggtactgatttcactttaaaaatttcacgtgttgaagctgaagatttaggagtatacttttgttcacaatcaacacacgttccttggacattaggtggtggtacaaaattagaaattaaacgtacggttgctgctccatcagtttttatttttccaccatcagatgaacaattaaaatcaggtactgcttcagttgtttgtttattaaataatttttatccacgtgaagctaaagttcaatggaaagttgataatgctttacaatcaggtaattcacaagaatcagttactgaacaagattcaaaagattcaacttattcattatcatcaactttaactttatcaaaagctgattatgaaaaacataaagtttatgcttgtgaagttactcatcaaggtttatcatcaccagttactaaatcatttaatcgtggtgaatgttaatctaga

SEQ ID NO: 8 - protein, 242 aa, Artificial Sequence
  Chimeric human kappa light chain
MDYKDHDGDYKDHDIDYKDDDDKDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTLGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1395 bp, Artificial Sequence
  Chimeric human heavy chain
atggaagttaaattagttgaatcaggtggtggtttagttaaaccaggaggttcattaaaattatcttgtgctgcttctggttttacattttcttcttatgctatgtcatgggttcgtcaaactcctgaaaaacgtttagaatgggtagcatcaatttcatcaggtggttcaacttattatcctgattctgtaaaaggtcgtttcactattttccgtgataatgcacgtaatattttatacttacaaatgtcttctttacgttcagaagatacagctatgtactattgtgtacgtggtcaagactattggggtcaaggtacatcagtaacagtatcagctagcacaaaaggtccatctgttttcccattagcaccatcatcaaaaagtacatctggtggtacagctgcattaggttgtttagttaaagactacttcccagaacctgtaactgtatcttggaactcaggtgcattaacaagtggtgtacacacattccctgctgttttacaatcatcaggtttatactcattaagtagtgtagtaacagtaccatcaagtagtttaggtacacaaacatacatctgtaacgtaaatcacaaaccaagtaacacaaaagtagacaaaaaagttgaaccaaaatcttgtgataaaacacacacttgtcctccatgtcctgctcctgaattattaggaggaccatcagtatttttattcccaccaaaacctaaagatacattaatgatttcacgtacacctgaagttacttgtgtagtagtagatgtttcacatgaagatccagaagttaaatttaattggtatgttgatggagtagaagttcacaatgcaaaaactaaaccacgtgaagaacaatataattctacatatcgtgtagtttcagttttaactgttttacaccaagattggttaaatggtaaagaatataaatgtaaagtatcaaataaagcattacctgcaccaattgaaaaaactatttcaaaagcaaaaggacaacctcgtgaaccacaagtttatacattaccaccatctcgtgatgaattaacaaaaaatcaagtatcattaacttgcttagtaaaaggtttctatccatcagatattgctgttgaatgggaatcaaatggtcaaccagaaaataattataaaacaactcctccagttttagattcagatggatcattcttcttatattcaaaattaacagtagataaatctcgttggcaacaaggaaatgttttctcatgttctgtaatgcacgaagctttacacaatcactatacacaaaaatctttatctttatcacctggtaaagactataaagatcacgatggtgattataaagaacacgatattgattacaaagatgatgatgataaataa

SEQ ID NO: 10 - protein, 464 aa, Artificial Sequence
  Chimeric human heavy chain
MEVKLVESGGGLVKPGGSLKLSCAASGFTFSSYAMSWVRQTPEKRLEWVASISSGGSTYYPDSVKGRFTIFRDNARNILYLQMSSLRSEDTAMYYCVRGQDYWGQGTSVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDYKDHDGDYKEHDIDYKDDDDK

(19) United States
(12) Patent Application Publication
(10) Pub. No.: US 2011/0151515 A1
(43) Pub. Date: Jun. 23, 2011

(54) PRODUCTION OF FC-FUSION POLYPEPTIDES IN EUKARYOTIC ALGAE

(75) Inventors: PETER HEIFETZ, San Diego, CA (US); Scott Franklin, Daly City, CA (US)

(73) Assignee: SAPPHIRE ENERGY, INC., SAN DIEGO, CA (US)

(21) Appl. No.: 12/777,952

(22) Filed: May 11, 2010

Related U.S. Application Data
(63) Continuation of application No. PCT/US2008/083225, filed on Nov. 12, 2008.
(60) Provisional application No. 60/987,734, filed on Nov. 13, 2007.

(51) Int. Cl. C12P 21/06; C12N 15/63; C12N 5/10; C12N 1/13

(52) U.S. Cl. 435/69.51; 435/69.7; 435/69.6; 435/69.52; 435/320.1; 435/419; 435/257.2

(57) ABSTRACT

Methods and compositions are disclosed to engineer plastids comprising heterologous genes encoding immuno-activating domains fused to an extracellular domain (ECD) of a receptor or surface glycoprotein, a growth factor or an enzyme and produced within a subcellular organelle, such as a chloroplast. The immuno-activating domains may include those regions of a protein capable of modulating the interaction between immune effector cells via proteins containing stereoselective binding domains and specific ligands, such as the Fc regions of antibodies. The present disclosure also demonstrates the utility of plants, including green algae, for the production of complex multi-domain fusion proteins as soluble bioactive therapeutic agents.

### CROSS REFERENCE TO RELATED APPLICATIONS

This application is a national phase filing of International Patent Application PCT/US2008/083225 entitled PRODUCTION OF FC-FUSION POLYPEPTIDES IN EUKARYOTIC ALGAE filed Nov. 12, 2008 which claims priority to and the benefit of U.S. Provisional Patent Application No. 60/987,734, entitled PRODUCTION OF FC-FUSION POLYPEPTIDES IN EUKARYOTIC ALGAE, filed Nov. 13, 2007, each of which is incorporated herein by reference in its entirety for all purposes.

### BACKGROUND

1. Field

The present invention relates generally to methods and compositions for expressing and purifying polypeptides manufactured in plastid (chloroplast) organelles, and more specifically to Fc-fusion constructs that encode therapeutic products that are expressed in chloroplasts.

2. Background Information

As the clinical success of recombinant protein-based therapies continues to grow, pharmaceutical companies are faced with several dilemmas, stemming primarily from increasing capital and production costs that threaten to limit the availability of these agents, as well as the molecules' increasing complexity. The inherently high cost of goods (COGs) for these molecules further exacerbates the fact that many of these proteins and monoclonal antibodies (mAbs) are used to treat chronic diseases that require multiple grams per patient, per year. Efficient expression and accumulation of biologically active eukaryotic proteins in their native, functional conformations is difficult to achieve at reasonable cost in cell-based biomanufacturing systems. In general, such platforms are either optimized for soluble protein secretion from highly capital-intensive mammalian cell cultures, or bacterial expression utilizing purified inclusion bodies that require inefficient downstream denaturation and refolding. The availability of less costly and more flexible manufacturing platforms for soluble, complex proteins is thus a significant unmet need. Patients, insurers, for-profit health maintenance organizations (HMOs) and the federal government, are also becoming increasingly reticent to pay for therapies that in some cases may exceed $50,000 per year.

Currently, there are a number of heterologous protein expression systems available for the production of therapeutic proteins for use both in human and animal healthcare. Each of these systems has distinct attributes in terms of protein yield, ease of manipulation, and cost of production. mAbs and complex therapeutic proteins are produced primarily by culture of transgenic mammalian cells in fixed bioreactor facilities consisting of scale up trains with stainless-steel vessels of increasing capacity, complex systems for media delivery and liquid handling, validatable clean-in-place systems, and equipment for upstream unit operations to deliver clarified extracts for further downstream processing. Due to high capital and media costs, and the inherent complexity of mammalian production systems, mAbs produced in this manner are very expensive, ranging from $150 to $1,000 per gram, prior to purification. Yeast and bacterial systems, while more economical in terms of media components, have several shortcomings in terms of protein expression, including an inability to efficiently produce properly folded functional molecules, as well as poor soluble yields of more complex proteins.

Some of the proteins that are difficult to produce in microbial systems include those incorporating fragment-crystallizable (Fc) domains derived from mammalian immunoglobulins. Fc domains are very useful, both with regard to incorporation into chimeric therapeutic proteins that have improved pharmacokinetic properties and long circulating half-lives, as well as their ability to facilitate protein purification using Protein A or Protein G affinity matrices. These latter reagents are well-known in the art as components of efficient capture resins that are useful for isolation of Fc-containing molecules from complex mixtures. As Fc domains are poorly suited for expression in bacterial systems it is desirable to evaluate their usefulness for enhancing purification efficiency in eukaryotic platforms capable of soluble expression of Fc-containing complex molecules. These Fc domains can also be removed from the final product if desired by incorporation of an enzyme-cleavable linker in the chimeric protein. For some protein products, incorporation of an Fc domain into the product can confer a therapeutic benefit.

Perhaps no other mAb-based therapy better illustrates the cost and scale dilemma than Omalizumab also known as Xolair. Xolair is a recently approved anti-IgE mAb therapy for allergic asthma. Xolair binds to the Fc portion of circulating IgE that normally interacts with FcεRIα, the high affinity receptor for IgE on the surface of mast cells and basophils. By preventing FcεRIα/IgE interaction, Xolair limits the production of inflammatory mediators produced by these cells (i.e. histamine, leukotrienes, protaglandins, and the like).

The cost of Xolair treatment varies from between $5,000-$12,000 per patient per year, depending upon patient weight and circulating levels of IgE at the time of treatment initiation. In North America alone, there are approximately fifteen million asthma sufferers, of whom, approximately 60%, or nine million, suffer from allergic asthma. Thus, to treat even 10% of the affected individuals in North America would cost between $4.5 and $10.8 billion dollars annually depending upon dosage requirements, while the amount of mAb required would be 1.8 to 9 metric tons. For comparison, in 2002, total mAb-based therapy production was only 916 kg while sales were around $ 5.2 billion. Clearly, a production platform that can substantially reduce both COGs and capitalization costs, and at the same time hold out the promise of treating substantially more afflicted individuals would be of tremendous benefit to the healthcare system in general.

Like Xolair and other anti-IgE mAbs, the naturally occurring receptor FcεRIα, binds IgE with high affinity. The human FcεRIα is a tetrameric complex located on the surface of mast cells and basophils, eosinophils, platelets and megakaryocytes. In these cell types, the receptor is comprised of an α chain, containing the ligand binding site, a β chain and a dimer of inter-molecularly, disulphide linked, γ chains, linked by inter-molecular disulphide bonds which are critically important for receptor accumulation. β chains are absent from this complex on the surface of both Langerhans cells and activated monocytes. The cytoplasmic tail and transmembrane domain of FcεRIα are not required for ligand binding and the extracellular domain of FcεRIα (ecFcεRIα) does not require glycosylation for ligand binding.

Recent success in the expression of recombinant proteins from both the nuclear and chloroplast genomes in the green alga Chlamydomonas reinhardtii serves to highlight the potential of algae for the production of recombinant proteins. The chloroplast organelle of algae and plants also is a particularly favorable site for recombinant protein production. Due to the presence of chaperones and foldases required for correct assembly of complex eukaryotic proteins imported from the cytosol, the chloroplast is well-suited for production of mammalian proteins that are both soluble and correctly folded. Algal-based production systems also offer tremendous advantages over traditional, as well as many alternative, expression platforms when it comes to speed, scalability, COGs and perhaps most importantly capital costs. However, despite demonstration of the feasibility of expressing human antibodies containing Fc domains in the chloroplasts of microalgae, no certainty was afforded that algae and their chloroplast organelles were amenable to expression and accumulation of chimeric or non-antibody molecules containing mammalian Fc domains.

### SUMMARY

The present invention discloses a method using plastid (chloroplast) organelle expression to generate fusion proteins that incorporate immunoglobulin-derived Fc domains suitable for conferring improved serum stability to therapeutic proteins. In addition, the method provides for efficient isolation and purification of such fusion proteins from complex extracts. These fused molecules may modulate the interaction between proteins containing stereoselective binding domains and specific ligands. The present invention also discloses nucleic acid constructs encoding such fusion proteins and the use of these fusion proteins in the treatment of various disorders, including inflammatory, metabolic and proliferative disorders. By incorporating an Fc domain capable of interacting with the neonatal FcRn receptor, it is possible to extend the pharmacokinetics of therapeutic proteins, thus minimizing adverse reactions caused by high doses, decreasing the frequency of injection, maximizing trancytosis to specific tissue sites, and decreasing production costs by reducing the dosage required to gain a useful pharmacological effect. In addition, expression of Fc-fusion proteins in the chloroplast results in the absence of asparagine-linked glycosylation on the Fc domain, thus prevents unwanted or undesired activation of Fc-mediated effector functions.

In one embodiment, a nucleic acid construct is disclosed including, in operable linkage, nucleic acid signaling elements for homologous recombination and expression of the fusion protein in a plant or algae plastid and a first polynucleotide sequence encoding a polypeptide including an Fc region and a second polynucleotide sequence encoding an extracellular domain (ECD) of a receptor or surface glycoprotein, a growth factor, a cytokine, or an enzyme, such as a metabolic enzyme whose deficiency results in a clinical disease state, where the first and second polynucleotide sequences are expressed as a fusion protein.

In one aspect, the first polynucleotide encodes a non-plastid, non-plant, eukaryotic polypeptide. In another aspect, the first polynucleotide encodes an aglycosylated Fc domain, that mediates binding to the FcRN receptor and Protein A or Protein G, but not to Fc receptors that mediate opsonization, cell lysis, and degranulation of mast cells, basophils and eosinophils.

In another aspect, the extracellular domain (ECD) of a receptor includes, but is not limited to, a cytokine receptor ECD, immunoglobulin receptor ECD, a T-cell receptor ECD, a cluster of differentiation antigen receptor ECD, growth factor receptor ECD, tissue factor receptor ECD, blood factor receptor ECD. In another aspect, the enzyme is DNAse I, a lipase, a protease, an amylase, uricase, asparaginase, collagenase, streptokinase, urokinase, tissue plasminogen activator, thrombin, lactoferrin, lysozyme, adenosine deaminase, N-acetylgalactosamine-4-sulfatase, alpha galactosidase, beta galactosidase, alpha glucocerebrosidase, beta glucocerebrosidase, alpha glucosidase, or iduronidase.

In one aspect, the growth factors include, but are not limited to, TGF-β, G-CSF, GM-CSF, IGF1, NGF, BDNF, NT3, PDGF, EPO, TPO, myostatin, GDF9, bFGF, EGF, and HGF.

In another aspect, the receptor ECD is IL-17 receptor ECD, TNFα receptor ECD, high affinity IgE receptor ECD, low affinity IgE receptor ECD, a chain high affinity IL-4 receptor ECD, IL-5 receptor ECD, IL-13 receptor ECD, IL-2 receptor ECD, cytotoxic T-lymphocyte-associated antigen 4 (CTLA-4), receptor activator of nuclear factor κβ (RANK), and tissue factor.

In one aspect, the cytokines include, but are not limited to, IL-1a, IL-1b, IL-2, IL-3, IL-4, IL-5, IL-6, IL-7, IL-8, IL-9, IL-10, IL-12, IL-13, IL-14, IL-15, IL-17, INF-α, INF-β, INF-γ, MIP-1a, MIP-1b, TGF-β, RANTES, MCP-1, MCP-2, MCP-3, MCP-4, and PF-4.

In another aspect, the surface glycoproteins include, but are not limited to, integrins, immunoglobulin superfamily members, selectins, and cadherins. In a related aspect, the glycoprotein is the extracellular Ig-like domain of human myelin oligodendrocyte glycoprotein (MOG).

In one embodiment, a plant cell or algae cell or progeny thereof is disclosed which contains a construct, where the construct includes, in operable linkage, nucleic acid signaling elements for homologous recombination and expression of the Fc-fusion protein in a plant or algae plastid and a first polynucleotide sequence encoding an Fc domain and a second polynucleotide sequence encoding an extracellular domain (ECD) of a receptor or surface glycoprotein, a growth factor, a cytokine or an enzyme, where the first and second polynucleotide sequences are expressed as a fusion protein.

In another embodiment, a plant cell or algae cell plastid is disclosed which contains a construct which includes, in operable linkage, nucleic acid signaling elements for homologous recombination and expression of the Fc-fusion protein in a plant or algae plastid and a first polynucleotide sequence encoding an Fc domain and a second polynucleotide sequence encoding an extracellular domain (ECD) of a receptor or surface glycoprotein, a growth factor, a cytokine or an enzyme, where the first and second polynucleotide sequences are expressed as a fusion protein.

In one aspect, the plant cell, algae cell or progeny contains the first and second polynucleotides that are stably integrated into the plastid of the cell. In another aspect, a vector includes such a construct.

In one embodiment, a method of producing an Fc-fusion protein is disclosed, including contacting a plastid with one or more expression constructs, where the expression constructs include, in operably linkage, a nucleic acid signal element for homologous recombination and expression of the fusion protein in the plastid and a first polynucleotide sequence encoding an Fc domain and a second polynucleotide sequence encoding an extracellular domain (ECD) of a receptor or surface glycoprotein, a growth factor, cytokine or an enzyme, wherein the first and second polynucleotide sequences are expressed as a fusion protein, allowing the construct to integrate into the genome of the plastid, and expressing the fusion protein encoded by the construct.

In another embodiment, a method of producing an aglycosylated Fc-fusion protein is disclosed, including contacting a plastid with one or more expression constructs, where the expression constructs include, in operably linkage, a nucleic acid signal element for homologous recombination and expression of the fusion protein in the plastid and a first polynucleotide sequence encoding an Fc domain and a second polynucleotide sequence encoding an extracellular domain (ECD) of a receptor or surface glycoprotein, a growth factor, cytokine or an enzyme, wherein the first and second polynucleotide sequences are expressed as a fusion protein, allowing the construct to integrate into the genome of the plastid, and expressing the fusion protein encoded by the construct.

In one aspect, the plastid is in a plant cell or algae cell or progeny thereof.

In a related aspect, the method further includes isolating the expressed protein from the plastid.

In another aspect, the isolation method comprises capture of the Fc domain on Protein A or Protein G.

In one aspect, an Fc-fusion protein is functional in vivo, and may include, but is not limited to, an ecFcεRIα-Fc fusion protein.

In one aspect, an Fc-fusion protein is functional in vivo, and may include, but is not limited to, an IGF1-Fc fusion protein.

In another embodiment, a plastid is disclosed which includes a nucleic acid expression construct, where the construct includes, in operable linkage, nucleic acid signaling elements for homologous recombination and expression of the fusion protein in a plant or algae plastid and a first polynucleotide sequence encoding an Fc domain and a second polynucleotide sequence encoding an extracellular domain (ECD) of a receptor or surface glycoprotein, a growth factor, a cytokine or an enzyme, where the first and second polynucleotide sequences are expressed as a fusion protein. In a related aspect, the plastid is a chloroplast.

In one embodiment, microalgae, macroalgae or progeny thereof, contain a plastid, where the plastid includes a nucleic acid expression construct, where the construct includes, in operable linkage, nucleic acid signaling elements for homologous recombination and expression of the fusion protein in a plant or algae plastid and a first polynucleotide sequence encoding an Fc domain and a second polynucleotide sequence encoding an extracellular domain (ECD) of a receptor or surface glycoprotein, a growth factor, a cytokine or an enzyme, where the first and second polynucleotide sequences are expressed as a fusion protein.

In one aspect, the algae is Chlamydomonas reinhardtii.

In another aspect, the microalgae is Dunaliella, Ankistrodesmus, Botryococcus, Chlorella, Haematococcus, Scenedesmus, Volvox or Porphyridium.

In another embodiment, an isolated fusion protein is disclosed which is generated by the steps including contacting a plastid with one or more expression constructs, where the expression constructs include, in operably linkage, a nucleic acid signal element for homologous recombination and expression of the fusion protein in the plastid and a first polynucleotide sequence encoding an Fc domain and a second polynucleotide sequence encoding an extracellular domain (ECD) of a receptor or surface glycoprotein, a growth factor, a cytokine or an enzyme, wherein the first and second polynucleotide sequences are expressed as a fusion protein, allowing the construct to integrate into the genome of the plastid, and expressing the fusion protein encoded by the construct.

In one embodiment, a method of treating a eukaryotic cell is disclosed including contacting the eukaryotic cell with a fusion protein isolated from a plant cell or algae cell or a plant cell or algae cell plastid which contains a construct which includes, in operable linkage, nucleic acid signaling elements for homologous recombination and expression of the fusion protein in a plant or algae plastid and a first polynucleotide sequence encoding an Fc domain and a second polynucleotide sequence encoding an extracellular domain (ECD) of a receptor or surface glycoprotein, a growth factor, a cytokine or an enzyme, where the first and second polynucleotide sequences are expressed as a fusion protein.

In another embodiment, a method of modulating receptor mediated signaling and/or immunologic response is disclosed including treating animal or human cells with a therapeutically effective dose of the fusion protein which is generated by the steps including contacting a plastid with one or more expression constructs, where the expression constructs include, in operably linkage, a nucleic acid signal element for homologous recombination and expression of the fusion protein in the plastid and a first polynucleotide sequence encoding an Fc domain and a second polynucleotide sequence encoding an extracellular domain (ECD) of a receptor or surface glycoprotein, a growth factor, a cytokine or an enzyme, wherein the first and second polynucleotide sequences are ex pressed as a fusion protein, allowing the construct to integrate into the genome of the plastid, and expressing the fusion protein encoded by the construct.

### BRIEF DESCRIPTION OF THE DRAWINGS

FIG. 1 shows a stained gel analysis of chloroplast expressed Fc-fusion protein after capture by Protein A affinity chromatography.

FIG. 2 provides a nucleotide sequence (A; SEQ ID NO:1) and amino acid sequence (B; SEQ ID NO:2) of the soluble receptor-Fc fusion protein IgER1-Fc receptor fusion described in FIG. 9. The receptor domain in B is underlined, the hinge region is in red (bold) and the c-terminal FLAG tag is in Bold italics.

FIG. 3 graphically illustrates the generation of C. reinhardtii chloroplast transformants expressing mAbs.

FIG. 4 shows the expression of HSV8-lsc by SDS PAGE and Western blot. Left Panel: Expression of HSV8-lsc in E. coli and C. reinhardtii chloroplast. Proteins were separated into soluble and insoluble phases by centrifugation. The proteins were separated by SDS-PAGE and stained with Coomassie blue or blotted to nitrocellulose and hybridized with an antiflag antibody. Right Panel: The chloroplast expressed proteins were separated on SDS-PAGE, blotted to nitrocellulose and hybridized with an anti-flag antibody. The arrow in the non-reduced (no-βME) lane indicates a band corresponding to an assembled dimeric antibody of approximately 130 kDa.

FIG. 5 shows the accumulation of HSV8 scfv antibody. Left Panel: Accumulation of HSV8 scfv antibody throughout the light dark cycle when grown at either 10⁶ or 10⁷ cells per ml and illustrates the fact that the resulting scfv is completely soluble. Right Panel: ELISA assay of fourteen independent transformants expressing HSV8 scfv, and shows that all the scfvs are able to bind glycoprotein D, its target antigen, from crude extract with out purification. This panel also illustrates the limited (less than two fold) variability typically seen among chloroplast transformants.

FIG. 6 shows the simultaneous accumulation of HSV8 LC and HC proteins in the chloroplast of C. reinhardtii. Panel A, top, shows the psbH region in the recipient strain, psbHΔ, used in these transformants. Note the strain contains the aphA6 cassette driven by the psbA 5′ and rbcL 3′ UTRs. Transforming constructs 7, 9, 31, and 8 were constructed by replacing the BamHI fragment containing the aphA6 cassette with the corresponding divergent promoter constructs shown below. Panel B shows a western blot of four independent transformants with the HSV8 lsc expressing strain as a control. Note that the orientations of the HC and LC genes in the vector make both heavy chain and light chain protein, showing that both promoter-UTRs work well, and that orientation of the coding region does not impact expression using this vector.

FIG. 7 shows that C. reinhardtii chloroplast expressed IgAs assemble into full length mAbs. Total soluble protein from strain 9-4-4 seen in FIG. 5 was subjected to flag-affinity chromatography. Equal volumes of flow-thru, wash and elution tractions were run on SDS PAGE with and without no-βME as indicated. Crude extracts and elution fractions 1-5 in the presence of βME shows LC and HC proteins migrating as discrete bands of 26 and 53 kDa, respectively. In the absence of βME, however; elution fractions 1-5 clearly show a coalescence of these bands into a larger product of ca. 160 kDa, the size expected for the fully assembled IgA.

FIG. 8 (A) shows various C. reinhardtii chloroplast 5′ promoter/UTR fragments as BamHI/NdeI restriction fragments. ecFcεRIα-hIgG1 or ecFcεRIα genes, assembled in C. reinhardtii chloroplast codon bias are shown as NdeI/XbaI fragments. 3′ UTRs are shown as XbaI/BamHI fragments. Any resulting 5′ promoter/UTR driving ecFcεRIα-hIgG1 or ecFcεRIα followed by any 3′UTR can subsequently be cloned into either p322 (Franklin et al, 2002) or psbH vectors as a BamHI insert. (B) shows that the divergent promoter/UTR can drive simultaneous expression of either ecFcεRIα-hIgG1 or ecFcεRIα. Any 3′ UTR can be utilized in this construct so long as it can be cloned as an XbaI/BamHI fragment. As seen in the Figure, the resulting cassette consisting of divergent promoter/5′UTR driving two copies of either ecFcεRIα-hIgG1 or ecFcεRIα, followed by a 3′ UTR is clonable as a BamHI fragment. This entire cassette can be subsequently cloned into either p322 or p3Hb.

FIG. 9 shows an anti-FLAG western blot analysis of samples prepared from psbD line expressing the IgER1-Fc receptor fusion (A; SEQ ID NO:1 and SEQ ID NO:2). The transplastomic line was grown at two light fluxes, 6 or 58 μEinsteins, as indicated, to a density of 5 E⁶ cells per ml. 20 μg of total soluble protein was run in crude lysate lane, while 7.5 μl of sample was rune in the Flow Thru (FT), 15 μl in wash fraction and 3.75 μl in E1 fractions. The final concentration sample (E2-17) represents 25% of the final pooled sample. The full length IgER1-Fc receptor fusion has a theoretical MW of 48.7 kDa. FLAG tagged bacterial alkaline phosphatase (BAP FLAG) was run as an internal control.

FIG. 10 shows the nucleotide (A; SEQ ID NO:3) and amino acid (B; SEQ ID NO:4) sequences for the murine light chain encoding NT73. The FLAG tag, is indicated in bold italics (B). Nucleotide (C; SEQ ID NO:5) and amino acid (D; SEQ ID NO:6) sequences for the murine heavy chain encoding NT73. The FLAG Tag in D is indicated by bold italics.

FIG. 11 shows the purification of mIgG1 from C. reinhardtii chloroplasts. Alga expressed mIgG1 were electrophoresed on 4-20% Tris-Glycine gels and either stained with Coomassie Brilliant Blue (left panel) or subjected to western blot analysis using anti-FLAG antibody (right panel). Samples run on Coomassie stained gel represent 5 or 10% of the total concentrated lysates.

FIG. 12 shows the nucleotide (A; SEQ ID NO:7) and amino acid (B; SEQ ID NO:8) sequence for the chimeric human light chain encoding NT73. The FLAG Tag in B is indicated by bold italics. Nucleotide (C; SEQ ID NO:9) and amino acid (D; SEQ ID NO:10) for chimeric human heavy chain encoding NT73. The FLAG Tag in D is indicated by bold italics

FIG. 13 shows the purification of hIgG1 from C. reinhardtii chloroplasts. Alga expressed hIgG1 were electrophoresed on 4-20% Tris Glycine gels and either stained with Coomassie Brilliant Blue (left panel) or subjected to western blot analysis using anti-FLAG antibody (right panel). Samples run on Coomassie stained gels represent 5% of the total concentrated lysates.

### DETAILED DESCRIPTION

Before the present composition, methods, and treatment methodology are described, it is to be understood that this invention is not limited to particular compositions, methods, and experimental Conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Modulating the potential negative impacts of molecules involved in disease progression is central to most current therapies. One approach to controlling the negative effects of such molecules is to prevent their interaction with receptors or other molecules that are required for the manifestation of the diseased state. The development of monoclonal antibodies that bind to these molecules, preventing their interactions, is central to the mechanism of action for several currently marketed therapeutics (i.e., Humira, Remicade and the like).

Rather than developing therapeutics de novo, as is the case with monoclonal antibodies, an alternative, but equally viable approach utilizes soluble receptors fused to Fc regions (Typically hinge, C_{H}2-C_{H}3 domains of heavy chain hIgG1, 2, 3, 4 or IgA, IgE, IgM or IgD molecules) of monoclonal antibodies. These Fc regions may be native, or modified in ways that increase or decrease their affinity with specific Fc receptors. For example, modifications to the Fc region of hIgG1 molecules can increase their interaction with FcγRIII on effector cells, thereby modulating ADCC. Likewise, modifications to Fc regions on hIgG1 can impact their interactions with FcγRIIB, the inhibitory Fc receptor, on effector cells, again to modulate ADCC. Modifications such as elimination of glycosylation (as would be induced by chloroplast organelle expression) can serve to abrogate some receptor-mediated functions without affecting interaction with the neonatal FcRn receptor which binds to the correctly folded CH2-CH3 hinge region in a non-glycosylation dependent manner.

Such Fc-receptor fusion molecules provide high affinity “traps” for their ligands, titrating them out of solution, with a high degree of specificity. The Fc portion of these molecules imparts increased half life to these molecules through their increased size and recycling through the neonatal receptor as well as a standardized and potentially modifiable means of purification via Protein A or G affinity chromatography. In one aspect, the fusion protein comprises a cleavable linker between the Fc and the fusion/therapeutic protein domain in order to effect efficient recovery after protein capture.

Another application of Fc fusion proteins as disclosed is for increasing the potency of other, non-receptor, protein moieties, including, but not limited to, growth factors, granulocyte cytokines, and enzymes, which are fused to Fc regions. While not being bound by theory, this increase in potency may be conferred by several mechanisms, including, but not limited to, increasing molecular weight leading to oligomerization. Such oligomerization can result in decreased loss of the therapeutic moiety via renal filtration. Also, more favorable pharmacokinetics may be obtained, where the Fc moiety confers Fc-mediated recycling to the therapeutic moiety. In one embodiment, a nucleic acid construct is disclosed including, in operable linkage, nucleic acid signaling elements for homologous recombination and expression of the fusion protein in a plant or algae plastid and a first polynucleotide sequence encoding a first polypeptide and a second polynucleotide sequence encoding a soluble receptor, where the first and second polynucleotide sequences are expressed as a fusion protein.

In one embodiment, a nucleic acid construct is disclosed including, in operable linkage, nucleic acid signaling elements for homologous recombination and expression of the fusion protein in a plant or algae plastid and a first polynucleotide sequence encoding a polypeptide consisting essentially of an Fc region and a second polynucleotide sequence encoding an extracellular domain (ECD) of a receptor or surface glycoprotein, a growth factor, cytokine or an enzyme.

For example, the present invention discloses the expression of the extracellular domain of the high affinity soluble receptor of IgE, ecFcεRIα, alone and as a fusion protein with the hinge C_{H}2 and C_{H}3 domains of human IgG1. While not being bound by theory, such a fusion protein binds to circulating IgE, specifically at the point of interaction with the FcεRIα, and prevents cross linking of IgE molecules on the surface of mast cells and subsequent release of allergic response mediators, including, but not limited to, TNFα, IL4, IL13, histamine, leukotrienes, prostaglandins, as well as the proteases tryptase and chymase (Pawankar et al., 2003; Hart, 2001). Further, such a fusion protein has therapeutic applications, including but not limited to, the treatment of allergic asthma. Moreover, given the expected rapid clearance rate for ecFcεRIα, fusion with an antibody fragment increases the stability of the receptor domain in sera, making such a receptor domain a more efficacious agent. Also, as expression of ecFcεRIα has been shown to have higher affinity for IgE in an non-glycosylated form, expression of the properly folded but non-glycosylated protein in chloroplasts provides a cost effective means to produce such non-glycosylated forms of an ECD.

As used herein “extracellular domain (ECD)” means a part of a receptor that projects out of the membrane on the outside of the cell or organelle. If the polypeptide chain of a receptor crosses the bilayer several times, the external domain may comprise several extrinsic “loops” protruding from the hydrophobic membrane. By definition, a receptor's main function is to recognize and respond to a specific ligand, for example, a neurotransmitter or hormone (although certain receptors respond also to changes in transmembrane potential), and in many receptors these ligands bind to the extracellular domain. Typically, soluble forms of receptors consist essentially of the ECD of a transmembrane receptor. For example, such ECDs include, but are not limited to, IL-17 receptor ECD, TNFα receptor ECD, high affinity IgE receptor ECD, low affinity IgE receptor ECD, a chain high affinity IL-4 receptor ECD, IL-5 receptor ECD, IL-13 receptor ECD, and IL-2 receptor ECD. Amino acid sequences for these receptors are well known in the art and include, but are not limited to, respectively, AAB99730 (IL-17 receptor); NP_{—}001057 (TNF α receptor); Q01362 (IgE high affinity receptor); P06734 (IgE low affinity receptor); P24394 (α chain high affinity IL-4 receptor); NP_{—}000555 (IL-5 receptor); P31785 (IL-2 receptor γ chain), NP_{—}000408 (IL-2 receptor α chain), NP_{—}000869 (IL-2 receptor β chain); AAA58389 (CTLA-4); AAB86809 (RANK); and AAB26852 (tissue factor).

As used herein “surface glycoprotein” is a conjugated protein which projects out of the membrane on the outside of the cell or organelle containing a non-protein moiety which is a carbohydrate. The glycoprotein need not be in a transmembrane form when present on a cell, and may simply be anchored to the cell surface in the native state. For the present invention, glycoproteins are in a soluble form and include, but are not limited to, integrins, immunoglobulin superfamily members, selectins, and cadherins.

As used herein “cognate” is used in a comparative sense to refer to genetic elements that are typically associated with a specific reference gene. For example, for the Photosystem II (PSII) gene psbA (i.e., a specific reference gene), cognate genetic elements would include, but are not limited to, a psbA promoter, psbA 5′ UTR, and psbA 3′ UTR. Contrapositively, “non-cognate” would refer to genetic elements that are not typically related to a specific reference gene. For example, but not limited to, where a chimeric construct comprising a psbA promoter and psbD 5′ UTR is to be homologously recombined at a psbA site, the 5′ UTR in the construct would be non-cognate to psbA.

As used herein “nucleic acid signaling element” is used broadly herein to refer to a nucleotide sequence that regulates the transcription or translation of a polynucleotide or the localization of a polypeptide to which it is operatively linked. A nucleic acid signaling element can be a promoter, enhancer, transcription terminator, an initiation (start) codon, a splicing signal for intron excision and maintenance of a correct reading frame, a STOP codon, an amber or ochre codon, an IRES, an RBS, a sequence encoding a protein intron (intein) acceptor or donor splice site, or a sequence that targets a polypeptide to a particular location, for example, a cell compartmentalization signal, which can be useful for targeting a polypeptide to the cytosol, nucleus, plasma membrane, endoplasmic reticulum, mitochondrial membrane or matrix, chloroplast membrane or lumen, medial trans-Golgi cisternae, or a lysosome or endosome. Cell compartmentalization domains are well known in the art and include, for example, a peptide containing amino acid residues 1 to 81 of human type II membrane-anchored protein galactosyltransferase, the chloroplast targeting domain from the nuclear-encoded small subunit of plant ribulose bisphosphate carboxylase, or amino acid residues 1 to 12 of the presequence of subunit IV of cytochrome c oxidase (see, also, Hancock et al., EMBO J. 10:4033-4039, 1991; Buss et al., Mol. Cell. Biol. 8:3960-3963, 1988; U.S. Pat. No. 5,776,689). Inclusion of a cell compartmentalization domain in a polypeptide produced using a method of the invention can allow use of the polypeptide, which can comprise a protein complex, where it is desired to target the polypeptide to a particular cellular compartment in a cell.

As used herein “immuno-activating (IA) domain” means a region of a immuno-effector protein capable of modulating effector cells of the immune system through the interaction between proteins containing stereoselective binding domains and specific ligands. Such ligands include, but are not limited to, immunoglobulins, cytokines, growth factors, tissue factors, and the like. For example, IgE, IL-1α, IL-1β, IL-2, IL-3, IL-4, IL-5, IL-6, IL-7, IL-8, IL-9, IL-10, IL-12, IL-13, IL-14, IL-15, IL-17, INF-α, INF-β, INF-γ, MIP-1a, MIP-1b, TGF-β, RANTES, MCP-1, MCP-2, MCP-3, MCP-4, PF-4, G-CSF, GM-CSF, NGF, BDNF, NT3, PDGF, EPO, TPO, myostatin, GDF9, bFGF, EGF, and HGF represent such ligands. Amino acid sequences for these ligands are well known in the art and include, but are not limited to, respectively, GenBank Acc. Nos. AAD41753 (IgE heavy chain), CAD54753 (IgE light chain); CAA27448 (IL-1a); NP_{—}000567 (IL-1β); CAA073317 (IL-2); AAC08706 (IL-3); NP_{—}758858 (IL-4); NP_CAA09587 (IL-5); NP_{—}000591 (IL-6); NP_{—}000871 (IL-7); AAH13615 (IL-8); AAC17735 (IL-9); NP_{—}000563 (IL-10); 1F45_B (IL-12 Chain B), P29459 (IL-12 Chain A); NP_{—}002179 (IL-13); CAA86100 (IL-15); AAV41220 (IL-17); AAA52716 (INF-α); AAC41702 (INF-β); AAB59534 (INF-γ); 1B53_B (MIP-1α Chain B), 1B53_A (MIP-1α Chain A); NP_{—}002975 (MIP1b); AAA36735 (TGF-β); AAC03541 (RANTES); AAB29926 (MCP-1); CAA76341 (MCP-2); CAB51055 (MCP-3); AAB67307 (MCP4); P02776 (PF-4); CAA01319 (G-CSF); AAA52578 (GM-CSF); CAA37703 (NGF); CAA62632 (BDNF); AA107076 (NT3); AAB26566 (PDGF A Chain), P01127 (PDGF B Chain); AAF23134 (EPO); AAB33390 (TPO); NP_{—}005250; AAH96228 (GDF9); AAB21432 (bFGF); NP_{—}001954; and AAA64297 (HGF).

As used herein “cell surface target” means a polypeptide, carbohydrate, lipid or a combination thereof on the plasma surface of a cell. In one embodiment, such targets include clusters of differentiation (CD), including, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, Cd24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, and the like.

As used herein “multifunctional” means having at least two functions. For example, a fusion protein comprising a binding domain and a ECD would be bifunctional.

As used herein “progeny” means a descendant or offspring, as a child, plant or animal. For example, daughter cells from a transgenic algae are progeny of the transgenic algae.

As used herein “transgene” means any gene carried by a vector or vehicle, where the vector or vehicle includes, but is not limited to, plasmids and viral vectors.

In a related aspect, integration of chimeric constructs into plastid genomes includes homologous recombination. In a further related aspect, cells transformed by the methods of the present invention may be homoplasmic or heteroplasmic for the integration, wherein homoplastic means all copies of the transformed plastid genome carry the same chimeric construct.

As used herein, the term “modulate” refers to a qualitative or quantitative increase or decrease in the amount of an expressed gene product. For example, where the use of light increases or decreases the measured amount of protein or RNA expressed by a cell, such light modulates the expression of that protein or RNA. In one aspect, modulation of expression includes autoregulation, where “autoregulation” refers to processes that maintain a generally constant physiological state in a cell or organism, and includes autorepression and autoinduction.

In a related aspect, autorepression is a process by which excess endogenous protein or endogenous mRNA results in decreasing the amount of expression of that endogenous protein. In a further related aspect, reduction of endogenous protein synthesis will result in increased transgene expression. In one aspect, operatively linking non-cognate genetic elements (e.g., promoters) to the endogenous gene is used to drive low levels of endogenous protein expression. In another aspect, mutations are introduced into the endogenous gene sequence and/or cognate genetic elements to reduce expression of the endogenous protein.

As used herein, the term “multiple cloning site” is used broadly to refer to any nucleotide or nucleotide sequence that facilitates linkage of a first polynucleotide to a second polynucleotide. Generally, a cloning site comprises one or a plurality of restriction endonuclease recognition sites, for example, a cloning site, or one or a plurality of recombinase recognition sites, for example, a loxP site or an att site, or a combination of such sites. The cloning site can be provided to facilitate insertion or linkage, which can be operative linkage, of the first and second polynucleotide, for example, a first polynucleotide encoding a first 5′ UTR operatively linked to second polynucleotide comprising a homologous coding sequence encoding a polypeptide of interest, linked to a first 3′ UTR, which is to be translated in a prokaryote or a chloroplast or both.

In one embodiment, a chimeric construct is disclosed including a PSII reaction center protein gene promoter, PSII gene 5′ UTR, a multiple cloning site (MCS), and a PSII gene 3′ UTR, having the configuration:

PSII gene promoter-
PSII gene 5′ UTR-MCS-PSII gene 3′ UTR.

In a related aspect, the PSII gene UTRs are from different PSII genes and may include, but are not limited to, a psbD 5′ UTR and a psbA 5′ UTR.

In another related aspect, the PSII gene promoter is a psbA or psbD promoter and the 3′ UTR is a psbA 3′ UTR.

In one aspect, the PSII gene promoter and PSII gene 5′ UTR are from psbD. In another aspect, the PSII gene 3′ UTR is a psbA 3′ UTR.

As used herein, the term “Photosystem II reaction center” refers to an intrinsic membrane-protein complex in the chloroplast made of D1 (psbA gene), D2 (psbD gene), alpha and beta subunits of cytochrome b-559 (psbE and psbF genes respectively), the psbI gene product and a few low molecular weight proteins (e.g., 9 kDa peptide [psbH gene] and 6.5 kDa peptide [psbW gene]). In a related aspect, endogenous genes embrace chloroplast genes that exhibit autoregulation of translation, and include, but are not limited to, cytochrome f (i.e., C-terminal domain) and photosystem I reaction center genes (e.g., psaA, PsaB, PsaC; PsaJ).

As used herein, the term “operatively linked” means that two or more molecules are positioned with respect to each other such that they act as a single unit and effect a function attributable to one or both molecules or a combination thereof. For example, a polynucleotide encoding a polypeptide can be operatively linked to a transcriptional or translational regulatory element, in which case the element confers its regulatory effect on the polynucleotide similarly to the way in which the regulatory element would effect a polynucleotide sequence with which it normally is associated with in a cell.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition, except that the term “synthetic polynucleotide” as used herein refers to a polynucleotide that has been modified to reflect chloroplast codon usage.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available (e.g., Ambion, Inc.; Austin Tex.), as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997). The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994; Ecker and Crooke, BioTechnology 13:351360, 1995).

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

The term “recombinant nucleic acid molecule” is used herein to refer to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide, or can comprise an encoding nucleotide sequence and a regulatory element, particularly a PSII promoter operatively linked to a PSII 5′ UTR. A recombinant nucleic acid molecule also can be based on, but manipulated so as to be different, from a naturally occurring polynucleotide, for example, a polynucleotide having one or more nucleotide changes such that a first codon, which normally is found in the polynucleotide, is biased for chloroplast codon usage, or such that a sequence of interest is introduced into the polynucleotide, for example, a restriction endonuclease recognition site or a splice site, a promoter, a DNA origin of replication, or the like.

One or more codons of an encoding polynucleotide can be biased to reflect chloroplast codon usage. Most amino acids are encoded by two or more different (degenerate) codons, and it is well recognized that various organisms utilize certain codons in preference to others. Such preferential codon usage, which also is utilized in chloroplasts, is referred to herein as “chloroplast codon usage”. Table 1 (below) shows the chloroplast codon usage for C. reinhardtii (see US2004/0014174, published Jan. 22, 2004).

TABLE 1
Chloroplast Codon Usage in Chlamydomonas reinhardtii

| | | | |
|---|---|---|---|
| UUU 34.1*(348**) | UCU 19.4(198) | UAU 23.7(242) | UGU 8.5(87) |
| UUC 14.2(145) | UCC 4.9(50) | UAC 10.4(106) | UGC 2.6(27) |
| UUA 72.8(742) | UCA 20.4(208) | UAA 2.7(28) | UGA 0.1(1) |
| UUG 5.6(57) | UCG 5.2(53) | UAG 0.7(7) | UGG 13.7(140) |
| CUU 14.8(151) | CCU 14.9(152) | CAU 11.1(113) | CGU 25.5(260) |
| CUC 1.0(10) | CCC 5.4(55) | CAC 8.4(86) | CGC 5.1(52) |
| CUA 6.8(69) | CCA 19.3(197) | CAA 34.8(355) | CGA 3.8(39) |
| CUG 7.2(73) | CCG 3.0(31) | CAG 5.4(55) | CGG 0.5(5) |
| AUU 44.6(455) | ACU 23.3(237) | AAU 44.0(449) | AGU 16.9(172) |
| AUC 9.7(99) | ACC 7.8(80) | AAC 19.7(201) | AGC 6.7(68) |
| AUA 8.2(84) | ACA 29.3(299) | AAA 61.5(627) | AGA 5.0(51) |
| AUG 23.3(238) | ACG 4.2(43) | AAG 11.0(112) | AGG 1.5(15) |
| GUU 27.5(280) | GCU 30.6(312) | GAU 23.8(243) | GGU 40.0(408) |
| GUC 4.6(47) | GCC 11.1(113) | GAC 11.6(118) | GGC 8.7(89) |
| GUA 26.4(269) | GCA 19.9(203) | GAA 40.3(411) | GGA 9.6(98) |
| GUG 7.1(72) | GCG 4.3(44) | GAG 6.9(70) | GGG 4.3(44) |

*Frequency of codon usage per 1,000 codons.
**Number of times observed in 36 chloroplast coding sequences (10,193 codons).

The term “biased”, when used in reference to a codon, means that the sequence of a codon in a polynucleotide has been changed such that the codon is one that is used preferentially in chloroplasts (see Table 1). A polynucleotide that is biased for chloroplast codon usage can be synthesized de novo, or can be genetically modified using routine recombinant DNA techniques, for example, by a site directed mutagenesis method, to change one or more codons such that they are biased for chloroplast codon usage. As disclosed herein, chloroplast codon bias can be variously skewed in different plants, including, for example, in alga chloroplasts as compared to tobacco.

Table 1 exemplifies codons that are preferentially used in algal chloroplast genes. The term “chloroplast codon usage” is used herein to refer to such codons, and is used in a comparative sense with respect to degenerate codons that encode the same amino acid but are less likely to be found as a codon in a chloroplast gene. The term “biased”, when used in reference to chloroplast codon usage, refers to the manipulation of a polynucleotide such that one or more nucleotides of one or more codons is changed, resulting in a codon that is preferentially used in chloroplasts. Chloroplast codon bias is exemplified herein by the alga chloroplast codon bias as set forth in Table 1. The chloroplast codon bias can, but need not, be selected based on a particular plant in which a synthetic polynucleotide is to be expressed. The manipulation can be a change to a codon, for example, by a method such as site directed mutagenesis, by a method such as PCR using a primer that is mismatched for the nucleotide(s) to be changed such that the amplification product is biased to reflect chloroplast codon usage, or can be the de novo synthesis of polynucleotide sequence such that the change (bias) is introduced as a consequence of the synthesis procedure.

In addition to utilizing chloroplast codon bias as a means to provide efficient translation of a polypeptide, it will be recognized that an alternative means for obtaining efficient translation of a polypeptide in a chloroplast to re-engineer the chloroplast genome (e.g., a C. reinhardtii chloroplast genome) for the expression of tRNAs not otherwise expressed in the chloroplast genome. Such an engineered algae expressing one or more heterologous tRNA molecules provides the advantage that it would obviate a requirement to modify every polynucleotide of interest that is to be introduced into and expressed from a chloroplast genome; instead, algae such as C. reinhardtii that comprise a genetically modified chloroplast genome can be provided and utilized for efficient translation of a polypeptide according to a method of the invention. Correlations between tRNA abundance and codon usage in highly expressed genes is well known (Franklin et al., Plant J. 30:733-744, 2002; Dong et al., J. Mol. Biol. 260:649-663, 1996; Duret, Trends Genet. 16:287-289, 2000; Goldman et. al., J. Mol. Biol. 245:467-473, 1995; Komar et. al., Biol. Chem. 379:1295-1300, 1998). In E. coli, for example, re-engineering of strains to express underutilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into chloroplasts to complement rare or unused tRNA genes in a chloroplast genome such as a C. reinhardtii chloroplast genome.

Generally, the chloroplast codon bias selected for purposes of the present invention, including, for example, in preparing a synthetic polynucleotide as disclosed herein reflects chloroplast codon usage of a plant chloroplast, and includes a codon bias that, with respect to the third position of a codon, is skewed towards A/T, for example, where the third position has greater than about 66% AT bias, particularly greater than about 70% AT bias. As such, chloroplast codon biased for purposes of the present invention excludes the third position bias observed, for example, in Nicotiana tabacum (tobacco), which has 34.56% GC bias in the third codon position (Morton B R, J Mol Evol (1993) 37(3):273-80). In one embodiment, the chloroplast codon usage is biased to reflect alga chloroplast codon usage, for example, C. reinhardtii, which has about 74.6% AT bias in the third codon position.

In one embodiment, a method to produce multifunctional fusion polypeptides/proteins is disclosed. The term “polypeptides/protein” is used broadly to refer to macromolecules comprising linear polymers of amino acids which act in biological systems, for example, as structural components (e.g., Fc regions), enzymes, chemical messengers, receptors, ligands, regulators, hormones, and the like. In one aspect, a plant cell or algae cell or progeny thereof is disclosed which contains a construct, where the construct includes, in operable linkage, nucleic acid signaling elements for homologous recombination and expression of the bifunctional fusion protein in a plant or algae plastid and a first polynucleotide sequence encoding a first polypeptide and a second polynucleotide sequence encoding an ECD, where the first and second polynucleotide sequences are expressed as a fusion protein. In another aspect, the fusion protein may include stabilizing molecules or domains, such as low complexity linkers. Such stabilizing molecules may form tripartite structures, which may include a stabilizing domain-Fc domain-ECD-domain. In one aspect, a fusion protein may comprise one or more stabilizing domains. Such tripartite molecules may also contain a small molecule drug, including, but not limited to therapeutic compounds. In one aspect, the tripartite molecule may comprise a cleavage domain (e.g., but not limited to, TEV protease recognition site).

In a related aspect, such tripartite molecules may be encoded by a single polynucleotide. In another aspect, a functional Fc domain of the tripartite molecule may comprise multimers of subunits to form a multimeric complex, where the tripartite structure is encoded with a first subunit of a multimer. The second or third or more subunits of the multimeric complex may be encoded on separate polynucleotides. In one aspect, the second, third or more subunits are integrated into different sites in the chloroplast genome, where each integrated subunit encoding polynucleotide comprises separate recombinational targeting sequences, promoters/5′ UTR regulatory sequences, and 3′ UTR sequences. In one aspect, the multimeric complex comprises a heavy chain and a light chain of an complete antibody.

In one embodiment, such fusion protein comprise multiple binding domains for targeting multiple surface markers. In one aspect, the fusion protein includes one or more ECDs which target specific ligands.

In another aspect, such polypeptides/proteins would include functional protein complexes, such as antibodies. The term “antibody” is used broadly herein to refer to a polypeptide or a protein complex that can specifically bind an epitope of an antigen. Generally, an antibody contains at least one antigen binding domain that is formed by an association of a heavy chain variable region domain and a light chain variable region domain, particularly the hypervariable regions. An antibody generated according to a method of the invention can be based on naturally occurring antibodies, for example, bivalent antibodies, which contain two antigen binding domains formed by first heavy and light chain variable regions and second heavy and light chain variable regions (e.g., an IgG or IgA isotype) or by a first heavy chain variable region and a second heavy chain variable region (V_{HH} antibodies; see, for example, U.S. Pat. No. 6,005,079), or on non-naturally occurring antibodies, including, for example, single chain antibodies, chimeric antibodies, bifunctional antibodies, and humanized antibodies, as well as antigen-binding fragments of an antibody, for example, an Fab fragment, an Fd fragment, an Fv fragment, and the like. In a related aspect, a heterologous gene encodes a single chain antibody comprising a heavy chain operatively linked to a light chain.

In another related aspect, polynucleotides useful for practicing a method of the producing such antibodies can be isolated from cells producing the antibodies of interest, for example, B cells from an immunized subject or from an individual exposed to a particular antigen, can be synthesized de novo using well known methods of polynucleotide synthesis, can be produced recombinantly or can be obtained, for example, by screening combinatorial libraries of polynucleotides that encode variable heavy chains and variable light chains (see Huse et al., Science 246:1275-1281 (1989)) and can be biased for chloroplast codon usage, if desired (see Table 1). These and other methods of making polynucleotides encoding, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995)).

Polynucleotides encoding humanized monoclonal antibodies, for example, can be obtained by transferring nucleotide sequences encoding mouse complementarity determining regions (CDRs) from heavy and light variable chains of the mouse immunoglobulin gene into a human variable domain gene, and then substituting human residues in the framework regions of the murine counterparts. General techniques for cloning murine immunoglobulin variable domains are known (see, for example, Orlandi et al., Proc. Natl. Acad. Sci., USA 86:3833, 1989), as are methods for producing humanized monoclonal antibodies (see, for example, Jones et al., Nature 321:522, 1986; Riechmann et al., Nature 332:323, 1988; Verhoeyen et al., Science 239:1534, 1988; Carter et al., Proc. Natl. Acad. Sci., USA 89:4285, 1992; Sandhu, Crit. Rev. Biotechnol. 12:437, 1992; and Singer et al., J. Immunol. 150:2844, 1993).

The disclosed methods can also be practiced using polynucleotides encoding human antibody fragments isolated from a combinatorial immunoglobulin library (see, for example, Barbas et al., Methods: A Companion to Methods in Immunology 2:119, 1991; Winter et al., Ann. Rev. Immunol. 12:433, 1994). Cloning and expression vectors that are useful for producing a human immunoglobulin phage library can be obtained, for example, from Stratagene Cloning Systems (La Jolla, Calif.).

A polynucleotide encoding a human monoclonal antibody also can be obtained, for example, from transgenic mice that have been engineered to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas, from which polynucleotides useful for practicing a method of the invention can be obtained. Methods for obtaining human antibodies from transgenic mice are described, for example, by Green et al., Nature Genet. 7:13, 1994; Lonberg et al., Nature 368:856, 1994; and Taylor et al., Intl. Immunol. 6:579, 1994; and such transgenic mice are commercially available (Abgenix, Inc.; Fremont Calif.).

The polynucleotide also can be one encoding an antigen binding fragment of an antibody. Antigen binding antibody fragments, which include, for example, Fv, Fab, Fab′, Fd, and F(ab′)₂ fragments, are well known in the art, and were originally identified by proteolytic hydrolysis of antibodies. For example, antibody fragments can be obtained by pepsin or papain digestion of whole antibodies by conventional methods. Antibody fragments produced by enzymatic cleavage of antibodies with pepsin generate a 5S fragment denoted F(ab′)2. This fragment can be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see, for example, Goldenberg, U.S. Pat. No. 4,036,945 and U.S. Pat. No. 4,331,647; Nisonhoff et al., Arch. Biochem. Biophys. 89:230. 1960; Porter, Biochem. J. 73:119, 1959; Edelman et al., Meth. Enzymol. 1:422 (Academic Press 1967); Coligan et al., In Curr. Protocols Immunol., 1992, see sections 2.8.1-2.8.10 and 2.10.1-2.10.4). Alternatively, an isolated Fc region can be generated recombinantly by methods well known in the art.

Another form of an antibody fragment is a peptide coding for a single complementarity-determining region (CDR). CDR peptides can be obtained by constructing a polynucleotide encoding the CDR of an antibody of interest, for example, by using the polymerase chain reaction to synthesize the variable region from RNA of antibody-producing cells (see, for example, Larrick et al., Methods: A Companion to Methods in Enzymology 2:106, 1991). Polynucleotides encoding such antibody fragments, including subunits of such fragments and peptide linkers joining, for example, a heavy chain variable region and light chain variable region, can be prepared by chemical synthesis methods or using routine recombinant DNA methods, beginning with polynucleotides encoding full length heavy chains and light chains, which can be obtained as described above.

Single celled algae, like C. reinhardtii, are essentially water borne plants and as such can produce proteins in a very cost effective manner. In addition, algae can be grown in complete containment, and there are a number of companies around the world that have developed large scale production of algae as, human nutraceuticals or as a food source for farmed fish and other organisms. Capitalization costs for an algal production facility are much less than for other types of cell culture, mainly because of the nature of algae and it's ability to grow with minimal input, using CO₂ as a carbon source and sunlight as an energy source. Although in many ways algae are an ideal system for therapeutic protein production there are a number of technical challenges that need to be met before algae can be used as an efficient production platform. Among these challenges are developing vectors that allow for consistent high levels of protein expression, and facilitating purification of recombinant proteins from complex cell lysates. One aspect of the present invention is to utilize immunoglobulin Fc domains to enable efficient purification of fusion proteins from algal and algal chloroplast extracts.

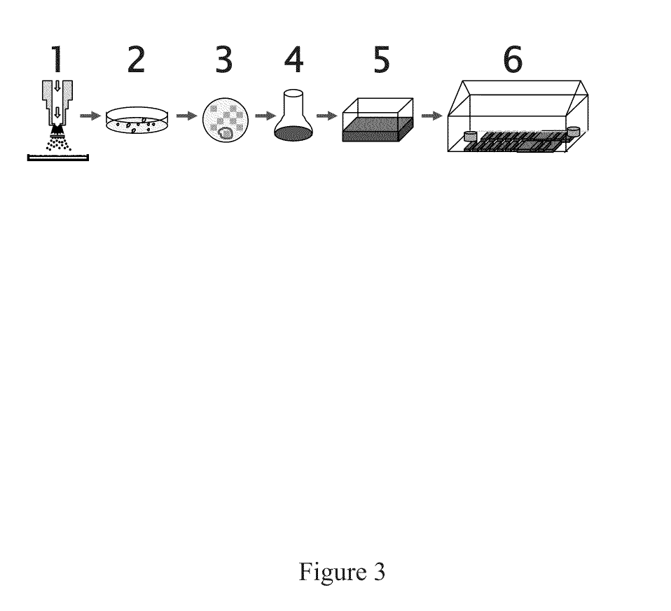

That such cells can be used to produce recombinant proteins is illustrated in FIG. 3. Large scale, closed systems as illustrated, currently operate for the production of nutraceuticals and animal feeds. Expression of proteins exclusively in the chloroplast of C. reinhardtii achieves far higher levels in this organelle than from genes transformed into the nucleus (Franklin et. al., 2002). For example, genes to be transformed into C. reinhardtii chloroplasts are first synthesized de novo in C. reinhardtii codon bias and cloned into chloroplast expression cassettes. Cells of C. reinhardtii, spread onto agar plates, are then transformed with chloroplast expression cassettes via biolistics (1). Colonies typically appear within ten days. Selection is provided either by co-transformation with markers conferring antibiotic resistance or rescue of phototrophy in strains deleted for essential photosynthetic genes. Such strains can be transformed with multiple cassettes, conferring both antibiotic resistance and rescue of phototrophy (2). Generation of homoplasmic lines (whereby all 50-80 copies of the chloroplast genome have converted to the recombinant genotype as determined by Southern blot analysis) typically requires seven additional days (3). Hence, within 24 days, stably transformed, homoplasmic lines expressing the desired protein of interest are obtained. In ten more days, multiple, twenty liter cultures, run in batch or continuous mode, can be ready for harvest (4). Hence, from gene assembly to production of milligram quantities of mAb requires as little as 34 days. Once larger scale cultures are required, scale up to 400 L systems would require an additional week (5), while moving up to multiple 30,000 L systems, would require another ten days (6). Given the observed accumulation levels for scFv, large single chain and full-length mAbs, the system as disclosed may go from gene to gram amounts of antibody in as little as 51 days. Further, this system has been applied to both C. reinhardtii and a separate species, Haematococcus pluvialis, a close relative of C. reinhardtii. Such cultures, at all of the indicated scales, may be run in batch, or semi-continuous mode. Cultures up to 20 L scale are easily run axenically in the lab, while larger cultures run at 400 to 30,000 L scale typically run for 60 days before requiring breakdown and cleaning in place.

A recombinant nucleic acid molecule useful in a method of the invention can be contained in a vector. The vector can be any DNA construct useful for introducing a polynucleotide into a chloroplast and, preferably, includes a nucleotide sequence of chloroplast genomic DNA that is sufficient to undergo homologous recombination with chloroplast genomic DNA, for example, a nucleotide sequence comprising about 400 to 1500 or more substantially contiguous nucleotides of chloroplast genomic DNA. Chloroplast vectors and methods for selecting regions of a chloroplast genome for use as a vector are well known (see, for example, Bock, J. Mol. Biol. 312:425-438, 2001; see, also, Staub and Maliga, Plant Cell 4:39-45, 1992; Kavanagh et al., Genetics 152:1111-1122, 1999).

The entire chloroplast genome of C. reinhardtii has been sequenced (Maul et al., Plant Cell

14(11):2659-79; GenBank Acc. No. BK000554). Generally, the nucleotide sequence of the chloroplast genomic DNA is selected for targeting such that it is not a portion of a gene, including a regulatory sequence or coding sequence, particularly a gene that, if disrupted due to the homologous recombination event, would produce a deleterious effect with respect to the chloroplast, for example, for replication of the chloroplast genome, or to a plant cell containing the chloroplast. In this respect, the Accession No. disclosing the C. reinhardtii chloroplast genome sequence also provides maps showing coding and non-coding regions of the chloroplast genome, thus facilitating selection of a sequence useful for constructing a vector of the invention. For example, the chloroplast vector, p322, which is used in experiments disclosed herein, is a clone extending from the Eco (Eco RI) site at about position 143. 1 kb to the Xho (Xho I) site at about position 148.5 kb.

The vector also can contain any additional nucleotide sequences that facilitate use or manipulation of the vector, for example, one or more transcriptional regulatory elements, a sequence encoding a selectable marker, one or more cloning sites, and the like. In one embodiment, the chloroplast vector contains a prokaryote origin of replication (ori), for example, an E. coli ori, thus providing a shuttle vector that can be passaged and manipulated in a prokaryote host cell as well as in a chloroplast.

The methods of the present invention are exemplified using the microalga, C. reinhardtii. The use of microalgae to express a polypeptide or protein complex according to a method of the invention provides the advantage that large populations of the microalgae can be grown, including commercially (Cyanotech Corp.; Kailua-Kona, Hi.), thus allowing for production and, if desired, isolation of large amounts of a desired product. However, the ability to express, for example, functional mammalian polypeptides, including protein complexes, in the chloroplasts of any plant allows for production of crops of such plants and, therefore, the ability to conveniently produce large amounts of the polypeptides.

In one embodiment, a method of expressing a chimeric gene is disclosed including transforming an algae cell by replacing an endogenous chloroplast gene via integration of a chimeric construct having a heterologous coding sequence, a promoter sequence, and at least one UTR, wherein the promoter is cognate or non-cognate to the endogenous chloroplast gene, and cultivating the transformed algae cell. In one aspect, a gene product encoded by the heterologous coding sequence is constitutively expressed. In a related aspect, the cells are homoplasmic for the integration.

In another embodiment, a method of expressing a chimeric gene includes transforming an algae cell by replacing psbA via integration of a chimeric construct comprising a nucleic acid sequence encoding the fusion protein as set forth in SEQ ID NO:2 or SEQ ID NO:4, a promoter sequence, and one or more UTRs, where the promoter is cognate or non-cognate to the endogenous chloroplast gene, and cultivating the transformed algae cell. In one aspect, at least two UTRs are psbA and psbD UTRs. In a related aspect, the nucleic acid sequence (e.g., SEQ ID NO:1 or SEQ. ID NO:3) is driven by a psbA or other promoter.

In one embodiment, an algae cell transformed by the methods of the invention is disclosed, where the algae includes, but is not limited to, Chlamydomonas reinhardtii.

Accordingly, the methods of the invention can be practiced using any plant having chloroplasts, including, for example, macroalgae, for example, marine algae and seaweeds, as well as plants that grow in soil, for example, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals such as azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum are also included. Additional ornamentals useful for practicing a method of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Leguminous plants useful for practicing a method of the invention include beans and peas. Beans include guar, locust bean, fenugreek, soybean, garden beans, cowpea, mung bean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop. Other plants useful in the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, chicory, groundnut and zucchini.

A method of the invention can generate a plant containing chloroplasts that are genetically modified to contain a stably integrated polynucleotide (i.e., transplastomes; see, for example, Hager and Bock, Appl. Microbiol. Biotechnol. 54:302-310, 2000; see, also, Bock, supra, 2001). The integrated polynucleotide can comprise, for example, an encoding polynucleotide operatively linked to a first and second UTR as defined herein. Accordingly, the present invention further provides a transgenic (transplastomic) plant, which comprises one or more chloroplasts containing a polynucleotide encoding one or more heterologous polypeptides, including polypeptides that can specifically associate to form a functional protein complex.

The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings; tubers, leaves, stems, fruit, seeds, roots, and the like. A part of a plant useful for propagation includes, for example, seeds, fruits, cuttings, seedlings, tubers, rootstocks, and the like.

A method of producing a heterologous polypeptide or protein complex in a chloroplast or in a transgenic plant of the invention can further include a step of isolating an expressed polypeptide or protein complex from the plant cell chloroplasts. As used herein, the term “isolated” or “substantially purified” means that a polypeptide or polynucleotide being referred to is in a form that is relatively free of proteins, nucleic acids, lipids, carbohydrates or other materials with which it is naturally associated. Generally, an isolated polypeptide (or polynucleotide) constitutes at least twenty percent of a sample, and usually constitutes at least about fifty percent of a sample, particularly at least about eighty percent of a sample, and more particularly about ninety percent or ninety-five percent or more of a sample. In one embodiment, an algal extract obtained from an algae cell transformed by replacing an endogenous chloroplast gene via integration of a chimeric construct having a heterologous coding sequence, a promoter sequence, and one or more UTRs, where the promoter is cognate or non-cognate to the endogenous chloroplast gene is disclosed.

The term “heterologous” is used herein in a comparative sense to indicate that a nucleotide sequence (or polypeptide) being referred to is from a source other than a reference source, or is linked to a second nucleotide sequence (or polypeptide) with which it is not normally associated, or is modified such that it is in a form that is not normally associated with a reference material. For example, a polynucleotide encoding an antibody is heterologous with respect to a nucleotide sequence of a plant chloroplast, as are the components of a recombinant nucleic acid molecule comprising, for example, a first nucleotide sequence operatively linked to a second nucleotide sequence, and is a polynucleotide introduced into a chloroplast where the polynucleotide is not normally found in the chloroplast.

The chloroplasts of higher plants and algae likely originated by an endosymbiotic incorporation of a photosynthetic prokaryote into a eukaryotic host. During the integration process genes were transferred from the chloroplast to the host nucleus (Gray, Curr. Opin. Gen. Devel. 9:678-687, 1999). As such, proper photosynthetic function in the chloroplast requires both nuclear encoded proteins and plastid encoded proteins, as well as coordination of gene expression between the two genomes. Expression of nuclear and chloroplast encoded genes in plants is acutely coordinated in response to developmental and environmental factors.

In chloroplasts, regulation of gene expression generally occurs after transcription, and often during translation initiation. This regulation is dependent upon the chloroplast translational apparatus, as well as nuclear-encoded regulatory factors (see Barkan and Goldschmidt-Clermont, Biochemie 82:559-572, 2000; Zerges, Biochemie 82:583-601, 2000; Bruick and Mayfield, supra, 1999). The chloroplast translational apparatus generally resembles that in bacteria; chloroplasts contain 70S ribosomes; have mRNAs that lack 5′ caps and generally do not contain 3′ poly-adenylated tails (Harris et al., Microbiol. Rev. 58:700-754, 1994); and translation is inhibited in chloroplasts and in bacteria by selective agents such as chloramphenicol.

Several RNA elements that act as mediators of translational regulation have been identified within the 5′UTR's of chloroplast mRNAs (Alexander et al., Nucl. Acids Res. 26:2265-2272, 1998; Hirose and Sugiura, EMBO J. 15:1687-1695, 1996; Mayfield et al., J. Cell Biol. 127:1537-1545, 1994; Sakamoto et al., Plant J. 6:503-512, 1994; Zerges et al., supra, 1997). These elements may interact with nuclear-encoded factors and generally do not resemble known prokaryotic regulatory sequences (McCarthy and Brimacombe, Trends Genet. 10:402-407, 1994).

A vector or other recombinant nucleic acid molecule of the invention can include a nucleotide sequence encoding a reporter polypeptide or other selectable marker. The term “reporter” or selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (Giacomin, Plant Sci. 116:59-72, 1996; Scikantha, J. Bacteriol. 178:121, 1996; Gerdes, FEBS Lett. 389:44-47, 1996; see, also, Jefferson, EMBO J. 6:3901-3907, 1997, fl-glucuronidase). A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain prokaryotic cells or plant cells or both that express the marker and, therefore, can be useful as a component of a vector of the invention (see, for example, Bock, supra, 2001). Examples of selectable markers include those that confer antimetabolite resistance, for example, dihydrofolate reductase, which confers resistance to methotrexate (Reiss, Plant Physiol. (Life Sci. Adv.) 13:143-149, 1994); neomycin phosphotransferase, which confers resistance to the aminoglycosides neomycin, kanamycin and paromycin (Herrera-Estrella, EMBO J. 2:987-995, 1983), hygromycin phosphotransferase, which confers resistance to hygromycin (Marsh, Gene 32:481-485, 1984), trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (White et al., Nucl. Acids Res. 18:1062, 1990; Spencer et al., Theor. Appl. Genet. 79:625-631, 1990), a mutant EPSP-synthase, which confers glyphosate resistance (Hinchee et al., BioTechnology 91:915-922, 1998), a mutant acetolactate synthase, which confers imidazolione or sulfonylurea resistance (Lee et al., EMBO J. 7:1241-1248, 1988), a mutant psbA, which confers resistance to atrazine, diuron or DCMU (Smeda et al., Plant Physiol. 103:911-917, 1993), or a mutant protoporphyrinogen oxidase (see U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants (see, for example, Maliga et al., Methods in Plant Molecular Biology, Cold Spring Harbor Laboratory Press, 1995, page 39). Since a composition or a method of the invention can result in expression of a polypeptide in chloroplasts, it can be useful if a polypeptide conferring a selective advantage to a plant cell is operatively linked to a nucleotide sequence encoding a cellular localization motif such that the polypeptide is translocated to the cytosol, nucleus, or other subcellular organelle where, for example, a toxic effect due to the selectable marker is manifest (see, for example, Von Heijne et al., Plant Mol. Biol. Rep. 9: 104, 1991; Clark et al., J. Biol. Chem. 264:17544, 1989; della Cioppa et al., Plant Physiol. 84:965, 1987; Romer et al., Biochem. Biophys. Res. Comm. 196:1414, 1993; Shah et al., Science 233:478, 1986; Archer et al., J. Bioenerg Biomemb. 22:789, 1990; Scandalios, Prog. Clin. Biol. Res. 344:515, 1990; Weisbeek et al., J. Cell Sci. Suppl. 11: 199, 1989; Bruce, Trends Cell Biol. 10:440, 2000.

The ability to passage a shuttle vector of the invention in a prokaryote allows for conveniently manipulating the vector. For example, a reaction mixture containing the vector and putative inserted polynucleotides of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. The shuttle vector then can be introduced into plant cell chloroplasts, wherein a polypeptide of interest can be expressed and, if desired, isolated according to a method of the invention.

A polynucleotide or recombinant nucleic acid molecule of the invention, which can be contained in a vector, including a vector of the invention, can be introduced into plant chloroplasts using any method known in the art. As used herein, the term “introducing” means transferring a polynucleotide into a cell, including a prokaryote or a plant cell, particularly a plant cell plastid. A polynucleotide can be introduced into a cell by a variety of methods, which are well known in the art and selected, in part, based on the particular host cell. For example, the polynucleotide can be introduced into a plant cell using a direct gene transfer method such as electroporation or microprojectile mediated (biolistic) transformation using a particle gun, or the “glass bead method” (see, for example, Kindle et al., supra, 1991), vortexing in the presence of DNA-coated microfibers (Dunahay, Biotechniques, 15(3):452-458, 1993), or by liposome-mediated transformation, transformation using wounded or enzyme-degraded immature embryos (see Potrykus, Ann. Rev. Plant. Physiol. Plant Mol. Biol. 42:205-225, 1991).

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). Chloroplast transformation involves introducing regions of chloroplast DNA flanking a desired nucleotide sequence into a suitable target tissue; using, for example, a biolistic or protoplast transformation method (e.g., calcium chloride or PEG mediated transformation). Fifty by to three kb flanking nucleotide sequences of chloroplast genomic DNA allow homologous recombination of the vector with the chloroplast genome, and allow the replacement or modification of specific regions of the plastid genome. Using this method, point mutations in the chloroplast 16S rRNA and rps 12 genes, which confer resistance to spectinomycin or streptomycin, can be utilized as selectable markers for transformation (Newman et al., Genetics 126:875-888, 1990; Svab et al., Proc. Natl. Acad. Sci., USA 87:8526-8530, 1990; Staub and Maliga, supra, 1992), and can result in stable homoplasmic transformants, at a frequency of approximately one per 100 bombardments of target tissues. The presence of cloning sites between these markers provides a convenient nucleotide sequence for making a chloroplast vector (Staub and Maliga, EMBO J. 12:601-606, 1993), including a vector of the invention. Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Goldschmidt-Clermont, Nucleic Acids Res 19:4083-4389, 1991; Svab and Maliga, Proc. Natl. Acad. Sci., USA 90:913-917, 1993). Approximately 15 to 20 cell division cycles following transformation are generally required to reach a homoplasmic state. Plastid expression, in which genes are inserted by homologous recombination into all of the up to several thousand copies of the circular plastid genome present in each plant cell, takes advantage of the enormous copy number advantage over nuclear-expressed genes to permit expression levels that can readily exceed 10% of the total soluble plant protein.

A direct gene transfer method such as electroporation also can be used to introduce a polynucleotide of the invention into a plant protoplast (Fromm et al., Proc. Natl. Acad. Sci., USA 82:5824, 1985). Electrical impulses of high field strength reversibly permeabilize membranes allowing the introduction of the polynucleotide. Microinjection can be performed as described in Potrykus and Spangenberg (eds.), Gene Transfer To Plants (Springer Verlag, Berlin, N.Y. 1995). A transformed plant cell containing the introduced polynucleotide can be identified by detecting a phenotype due to the introduced polynucleotide, for example, expression of a reporter gene or a selectable marker.

Microprojectile mediated transformation also can be used to introduce a polynucleotide into a plant cell chloroplast (Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000 particle gun (BioRad; Hercules Calif.). Methods for the transformation using biolistic methods are well known (Wan, Plant Physiol. 104:37-48, 1984; Vasil, BioTechnology 11: 1553-1558, 1993; Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (Duan et al., Nature Biotech. 14:494-498, 1996; Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, the glass bead agitation method (Kindle et al., supra, 1991), and the like.

Reporter genes have been successfully used in chloroplasts of higher plants, and high levels of recombinant protein expression have been reported. In addition, reporter genes have been used in the chloroplast of C. reinhardtii, but, in most cases very low amounts of protein were produced. Reporter genes greatly enhance the ability to monitor gene expression in a number of biological organisms. In chloroplasts of higher plants, beta-glucuronidase (uidA, Staub and Maliga, EMBO J. 12:601-606, 1993), neomycin phosphotransferase (nptll, Carrer et al., Mol. Gen. Genet. 241:49-56, 1993), adenosyl-3-adenyltransferase (aadA, Svab and Maliga, Proc. Natl. Acad. Sci., USA 90:913-917, 1993), and the Aequorea victoria GFP (Sidorov et al., Plant J. 19:209-216, 1999) have been used as reporter genes (Heifetz, Biochemie 82:655-666, 2000). Each of these genes has attributes that make them useful reporters of chloroplast gene expression, such as ease of analysis, sensitivity, or the ability to examine expression in situ.

An Fc-fusion protein of the present invention may be used as a medicament to treat a subject, in need thereof, by administering an effective concentration of the protein in combination with a pharmaceutically acceptable carrier. In a related aspect, such Fc-fusion proteins may include, but are not limited to, an IL-17 receptor ECD-Fc fusion protein, a TNF α receptor ECD-Fc fusion protein, an FcεRIα receptor ECD-Fc fusion protein, an IL-4 receptor ECD-Fc fusion protein, an IL-5 receptor ECD-Fc fusion protein, an IL-13 receptor ECD-Fc fusion protein, an IL-2 receptor ECD-Fc fusion protein, a CTLA-4-Fc fusion protein, a RANK-Fc fusion protein. These Fc-receptor fusion proteins are useful for treating rheumatoid arthritis, Crone's disease, irritable bowel syndrome, asthma, hypereosinophilia, suppression of T-cell responses in transplantation, and 1in anti-resorptive therapy (e.g., tumor induced hypercalcemia).

In another aspect, the fusion proteins include, but are not limited to, a Factor-VII-Fc fusion protein, a Glucagon-like protein 1-Fc fusion protein, a G-CSF-Fc-fusion protein, a GM-CSF-Fc fusion protein, a DNAse I-Fc fusion protein, an IGF-1-Fc fusion protein, an EPO-Fc fusion protein, an interferon α-Fc fusion protein, an interferon β-Fc fusion protein, an interferon γ-Fc fusion protein, a MOG-Fc fusion protein and a CD24-Fc fusion protein. These fusion proteins are useful for treating autoimmune disorders (e.g., MS, diabetes), demyelinating diseases, age related macular degeneration, metabolic disorders, and can be useful in conjunction with bone marrow transplantation and chemotherapy regimens.

Effective concentrations of the compositions provided herein or pharmaceutically acceptable salts or other derivatives thereof are mixed with a suitable pharmaceutical carrier or vehicle. Derivatives of the compounds, such as salts of the compounds or prodrugs of the compounds may also be used in formulating effective pharmaceutical compositions. The concentrations of the compounds are effective for delivery of an amount, upon administration, that ameliorates the symptoms of the disease. Typically, the compositions are formulated for single dosage administration.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients.

The active compounds can be administered by any appropriate route, for example, orally, parenterally, intravenously, intradermally, subcutaneously, or topically, in liquid, semi-liquid or solid form and are formulated in a manner suitable for each route of administration. Preferred modes of administration include oral and parenteral modes of administration. The active compound is included in the pharmaceutically acceptable carrier in an amount sufficient to exert a therapeutically useful effect in the absence of undesirable side effects on the patient treated. In one aspect, treated may be performed by contacting cells with the fusion protein of the invention ex vivo.

The therapeutically effective concentration may be determined empirically by testing the compounds in known in vitro and in vivo systems as described herein or known to those of skill in this art and then extrapolated therefrom for dosages for humans.

The concentration of active compound in the drug composition will depend on absorption, inactivation and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, a fusion protein as set forth in SEQ ID NO: 2 or SEQ ID NO: 4 is disclosed, including fusion protein containing compositions admixed with pharmaceutically acceptable carriers.

In one aspect, a composition may include the fusion protein in combination with chemotherapeutic compounds, where such a combination may be used to treat a subject in need thereof. In one aspect, such chemotherapeutics include, but are not limited to, Aclacinomycins, Actinomycins, Adriamycins, Ancitabines, Anthramycins, Azacitidines, Azaserines, 6-Azauridines, Bisantrenes, Bleomycins, Cactinomycins, Carmofurs, Carmustines, Carubicins, Carzinophilins, Chromomycins, Cisplatins, Cladribines, Cytarabines, Dactinomycins, Daunorubicins, Denopterins, 6-Diazo-5-Oxo-L-Norleucines, Doxifluridines, Doxorubicins, Edatrexates, Emitefurs, Enocitabines, Fepirubicins, Fludarabines, Fluorouracils, Gemcitabines, Idarubicins, Loxuridines, Menogarils, 6-Mercaptopurines, Methotrexates, Mithramycins, Mitomycins, Mycophenolic Acids, Nogalamycins, Olivomycines, Peplomycins, Pirarubicins, Piritrexims, Plicamycins, Porfiromycins, Pteropterins, Puromycins, Retinoic Acids, Streptonigrins, Streptozocins, Tagafurs, Tamoxifens, Thiamiprines, Thioguanines, Triamcinolones, Trimetrexates, Tubercidins, Vinblastines, Vincristines, Zinostatins, and Zorubicins.

The tablets, pills, capsules, troches and the like can contain any of the following ingredients, or compounds of a similar nature: a binder, such as microcrystalline cellulose, gum tragacanth and gelatin; an excipient such as starch and lactose, a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, and fruit flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings and flavors. The active materials can also be mixed with other active materials which do not impair the desired action, or with materials that supplement the desired action.

The following examples are intended to illustrate but not limit the invention.

### EXAMPLES

### Materials and Methods

Synthesis of antibody and construction of Fc-fusion proteins.

Coding regions for all recombinant proteins were synthesized de novo in C. reinhardtii chloroplast codon bias (Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53).

As a means to achieve higher levels of recombinant protein accumulation the question of codon bias in the chloroplast was specifically addressed. Using a codon optimized gfp gene, accumulation of codon optimized GFP increased 80 fold as compared to a non-optimized GFP counterpart was demonstrated (Franklin et. al., 2002). The chloroplast codon optimized GFP, driven by the rbcL promoter and 5′ UTR, accumulated to 0.5% of total soluble protein. More recently, a chloroplast codon optimized LuxAB gene, luxCt, has been developed (Mayfield and Schultz, 2003) that allows for in situ visualization of recombinant protein accumulation. Collectively, these data established that algal chloroplasts have the capacity to synthesize fairly complex molecules in a soluble and active form.

The high levels of expression of the codon optimized gfpCt and luxCt genes lead to examination of high levels of human antibody expression in eukaryotic algae through codon optimization of human antibody genes. Because of the complexity of engineering a strain to express both a heavy and light chain gene simultaneously, an antibody gene was designed that could be expressed as a single coding region, but one that was more complex then a simple scFv antibody. For this, a large single chain anti Herpes Simplex virus antibody gene was engineered, HSV8, in C. reinhardtii chloroplast codon bias. This large single chain (lsc) antibody contained the entire IgA heavy chain fused to the variable region of the light chain by a flexible linker. This gene was designed to examine both expression and assembly, as this lsc antibody contains the Fc portion of the heavy chain, which is the site normally involved in intermolecular disulfide bond formation leading to dimerization of the antibody. C. reinhardtii chloroplast atpA or rbcL promoters and 5′ untranslated regions were used to drive expression.

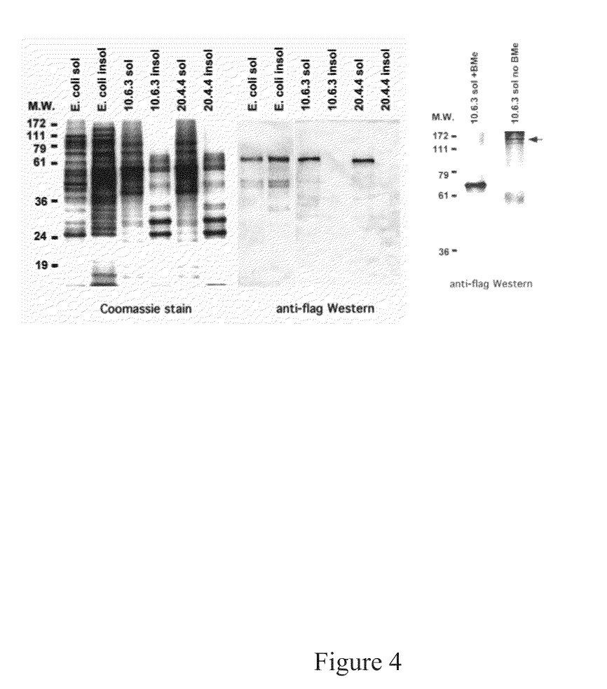

The HSV8 antibody is directed against Herpes Simplex virus glycoprotein D (Burioni et al 1994), and as shown in FIG. 4, expression of the HSV8-lsc antibody in E. coli results in the accumulation of protein mainly in the insoluble pellet. The small amount of HSV8-lsc that accumulates in the soluble phase of E. coli was unable to bind antigen. Expression of the HSV8-lsc protein in C. reinhardtii chloroplast results in the accumulation of a completely soluble protein, with no antibody found in the insoluble pellet (FIG. 4, central panel). The chloroplast expressed HSV8-lsc antibody was found to bind herpes virus proteins, as determined by ELISA assays, while no binding was detected from the E. coli expressed protein. The chloroplast expressed antibody was also found to assemble into higher order complexes that were susceptible to reduction by βME (FIG. 4, right panel), suggesting that the chloroplast expressed antibody is assembling into dimers of the correct size (ca. 130 kDa) in vivo. Formation of disulfide bonds in recombinant proteins expressed in chloroplast has previously been shown for human somatotropin expressed in tobacco chloroplast (Staub et al. 2000), and was somewhat expected given the presence of protein disulfide isomerase in algal chloroplasts (Kim and Mayfield 1997; Mayfield et al. 2003).

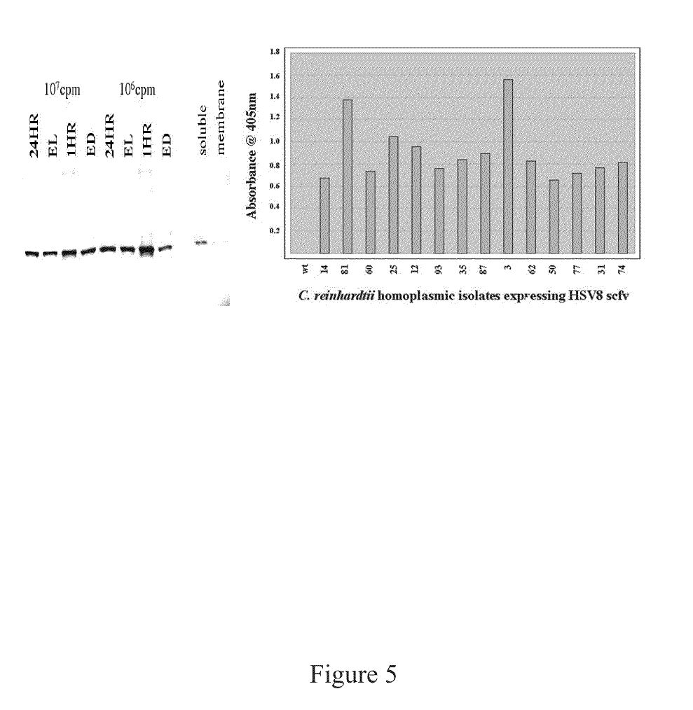

In addition to expressing the large single chain antibody, a conventional single chain antibody containing only the variable regions of the light and heavy chains fused together (scFv) was also expressed. As shown in FIG. 5 (left panel), the scFv accumulated in chloroplast in the soluble phase, and appears to be somewhat more stable than the HSV8-lsc protein. To test for binding activity the scFv was used in ELISA assays against HSV proteins. As shown in FIG. 5 (right panel) the scFv was able to bind HSV proteins, even from crude cell extracts without purification, demonstrating that the protein was correctly folded into a functional antigen binding protein.

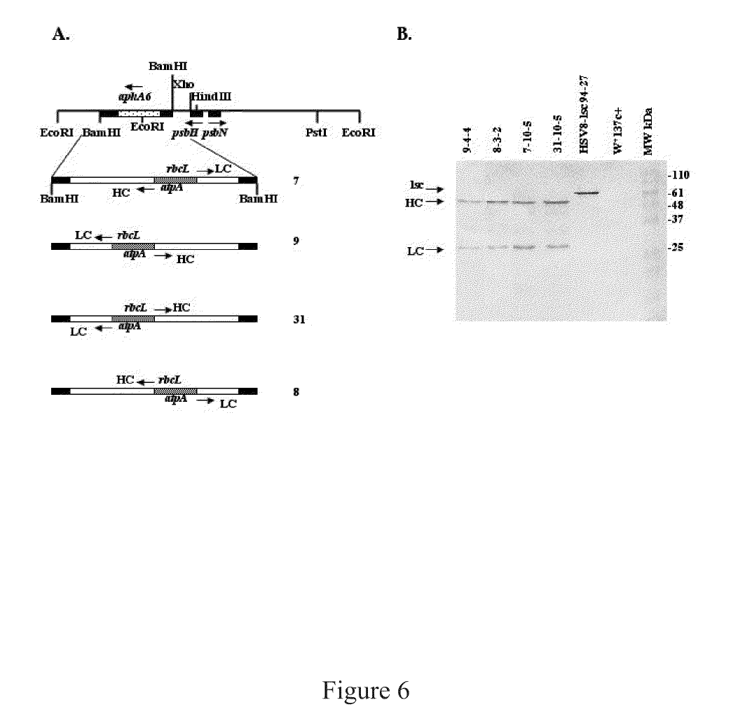

As shown in FIG. 6, expression of full length light chain and heavy chain proteins from a human IgA antibody has been achieved. In addition, a dual promoter-UTR cassette based upon the endogenous rbcL and atpA genes that allows for linked integration and simultaneous expression of two copies of a recombinant gene has been developed. As shown in FIG. 6A, the rbcL and atpA genes are transcribed from adjacent divergent promoters in the C. reinhardtii chloroplast genome and both promoters are contained within a segment of DNA spanning 863 bp. This divergent promoter has the advantage in that one need only select for a single integration event in order to integrate two copies of a gene. For expression of heavy chain and light chain, the HC and LC HSV8 genes, divergent promoter and 3′ UTR were ligated so that four separate constructs were made, with both genes being driven by each promoter in both possible orientations. Each of these constructs was placed into the p3HB chloroplast expression cassette. This cassette was developed to allow for integration events into a C. reinhardtii strain, psbHΔ, in which the psbH gene was disrupted through the introduction of a single nucleotide resulting in a frame shift. The introduction of the mutation was selected for by the inclusion of the adjacent aphA6 cassette conferring resistance to the antibiotic kanamycin (Bateman and Purton, 2000). Introduction of recombinant genes into strain psbHΔ, results in elimination of the aphA6 cassette and conversion of the mutated psbH gene to wild type with conversion to phototrophy. Hence, transformants are selected for based upon their ability to grow on minimal media. Constructs 7 and 9 both contain the light chain gene driven by the rbcL promoter-UTR and the heavy chain gene driven by the atpA promoter-UTR. Each coding region is followed by the rbcL 3′ UTR. Construct 7 is oriented such that the LC gene is in the reverse orientation with respect to the endogenous psbH gene, while in construct 9 it is in the forward orientation.

Constructs 8 and 31 contain the light chain gene driven by the atpA promoter-UTR and heavy chain gene driven by the rbcL promoter-UTR, again, each coding region is followed by the rbcL 3′ UTR. All four constructs were transformed into a psbH deletion strain and selected for growth on minimal media. As shown in FIG. 6 B, independent isolates of all four transgenic lines accumulate both the 27 kDa light chain protein and a 53 kDa heavy chain protein. Both heavy chain and light chain proteins accumulate well, and appear to be in stoichiometric amounts. These data thus demonstrate the utility of the divergent promoter for the simultaneous expression of two gene products, and show that chloroplasts accumulate full-length human antibody proteins as soluble proteins. This divergent promoter is used to increase expression levels of the IgE receptor and receptor fusion proteins.

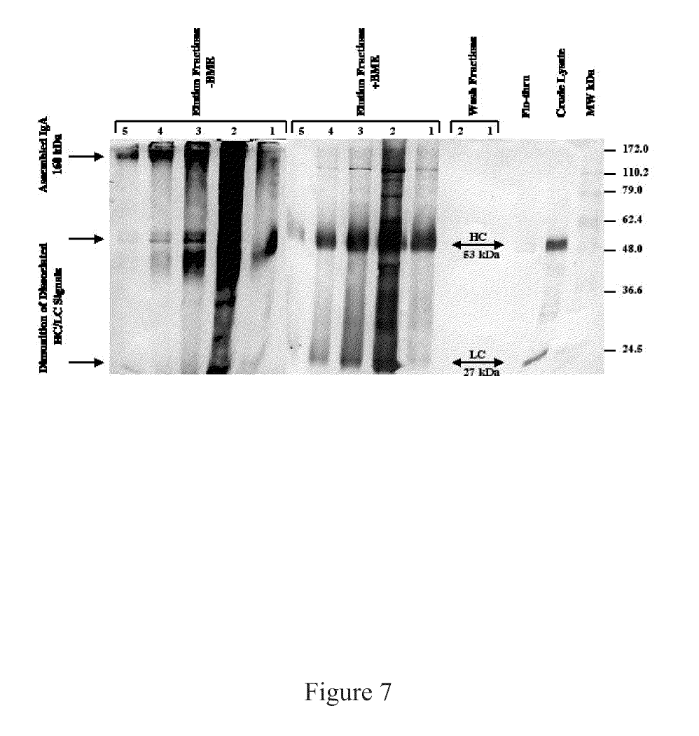

In addition, as seen in FIG. 7, FLAG affinity purified IgAs from strain 9-4-4 assemble into full length mAbs as visualized by SDS PAGE in the absence of βME. These mAbs were also shown to function in ELISA assays.

For the three examples presented above, HSV8 scfv, large single chain and the full-length IgA, the expression levels range from 0.2% of total soluble protein for the scfv molecule to 0.5% for the large single chain molecule. To put these numbers into perspective, see Table 2, below. In this table a loss of 50% of the recombinant protein on purification from crude extracts was assumed.

Table 2 showing the accumulation of soluble protein relative to biomass in cells grown at laboratory scale.

| | |
|---|---|
| Biomass/L cells: | 1 g |
| Total soluble protein/L: | 0.25 g |

| Unpurified mAbs/L at: | mg/L | For 10 mg purified |
|---|---|---|
| 0.25% tsp | 0.625 | 32.0 L |
| 0.5% tsp | 1.25 | 16.0 L |
| 1% tsp | 2.5 | 8.0 L |
| 3% tsp | 7.5 | 2.67 L |
| | | (Assumes 50% yield) |

Numbers assume a cell density of 1.0×10⁷ cell ml⁻¹. For large scale cultures (in excess of 400 L) cell counts and hence biomass and soluble protein, generally are 50% of the values shown here. These latter values were used in calculating yield data discussed for a large scale facility.

cDNAs encoding the ecFcεRIα alone, excluding the ER targeting sequence, with and without a FLAG epitope at the C-terminus and as a fusion with human IgG1 hinge, C_{H}2 and C_{H}3 domains (either with or without a FLAG epitope at the C-terminus of the IgG1 moiety) are synthesized in C. reinhardtii chloroplast codon bias utilizing a PCR-based approach (Stemmer et. al., 1995) as previously described (Franklin et. al., 2002). The cDNA sequence of human ecFcεRIα is based on Gen Bank Accession no. X06948 and excludes the transmembrane spanning domain and cytoplasmic tail. The nucleotide sequence spans residues 182 to 718 resulting in a peptide 180 aa in length.

Although epitope tags such as FLAG can greatly aid in protein purification, such epitope tagged molecules are not considered pharmacologically acceptable for use in animals or humans. Epitope tags do, however, facilitate rapid and highly sensitive detection and quantification of recombinant protein accumulation levels with which to aid in following the molecules as they are expressed in situ.

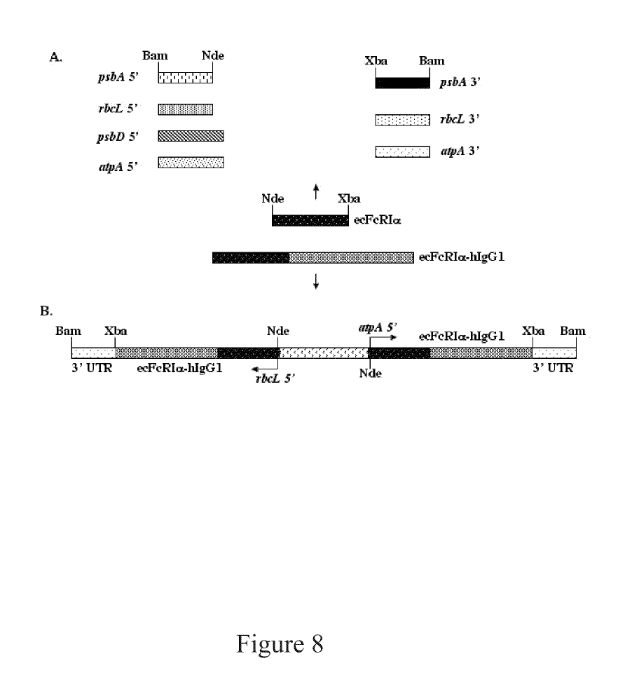

C. reinhardtii chloroplast codon optimized Flag tagged and native ecFcεRIα and its fusion with hIgG1, both Flag tagged and native, are synthesized with Nde I and Xba I restriction sites at the 5′ and 3′ ends respectively. Gene assembled products are first cloned into pCR Topo 2.1 vectors to confirm that the assembled products are of the correct sequence. Any point mutations are repaired using site-directed mutagenesis. Constructs having the correct nucleotide sequence are then cloned directly into the chloroplast transformation construct p322 (Franklin et. al., 2002), as a Bam HI restriction fragment when furnished with the appropriate 5′ promoter/UTR and 3′UTR as illustrated in FIG. 8A. At the same time, constructs with the correct sequence can be cloned into pBAD/gIII (Invitrogen) which has been modified to accept Nde I/Xba I inserts for soluble expression in E. coli.

Accumulation of various recombinant proteins in the C. reinhardtii chloroplast is greatly affected by the choice of 5′ promoter/UTR and less so by the 3′ UTR. Thus, for the expression of ecFcεRIα and its fusion with hIgG1, all of the 5′ promoter/UTRs listed in FIG. 8A are tested.

Transformation of C. reinhardtii chloroplast with p322 constructs is carried out in strain 137 c+ via co-transformation with plasmid p228 which confers spectinomycin resistance by virtue of a point mutation in the 16S rRNA gene (Franklin et. al., 2002). Positive transformants are selected on TAP media plus spectinomycin (Harris, 1989) and subjected to two or more rounds of streaking on selective media to obtain homoplasmic lines as determined by Southern blot analysis. Purification of FLAG-tagged ecFcεRIα-hIgG1 and ecFcεRIα from C. reinhardtii expressing strains is carried out by anti-FLAG affinity chromatography as described (Mayfield and Franklin 2003). Purification of the non-epitope, Flag tagged receptor-IgG1 fusion and non-epitope tagged soluble receptor, is carried out using Protein A chromatography.

Integration of constructs at the p322 site results in two copies of the insert per chloroplast genome, as this region lies in the duplicated inverted repeat region. Additionally, a divergent promoter/5′UTR cassette shown in FIG. 7B is provided. This construct may integrate up to six copies of either ecFcεRIα or its fusion with hIgG1 per chloroplast genome by co-transforming the divergent promoter/5′UTR cassette into a psbHΔ strain (FIG. 6) at both the p322 site (selection provided by co-transformation with spectinomycin resistance plasmid, p228) and the psbH site (selection provided by rescue of the psbH gene and ability to grow on acetate free medium).

hIgE is used as a substrate in ELISAs, as a purification reagent for recombinantly expressed ecFcεRIα and as a reagent in cell based assays. The human IgE (hIgE) JW8, specific for the hapten 4-hydroxy-3-nitrophenylacetyl (NIP), is purified from the transfectoma JW8/5/13 (European Cell Culture Collection, ref. no. 87080706) as described by Nechansky et al. (2001) over a NIP-BSA column. JW8/5/13 is propagated by conventional methods and JW8 is purified over NIP-BSA affinity matrices. JW8 purified in this fashion serves as a substrate in ELISAs to assess the binding of receptor and receptor mAb fusions to IgE.

In addition, an ecFcεRIα affinity column is optionally constructed using purified JW8. JW8 purified as described above, is coupled to cyanogen bromide-activated sepharose. This affinity column is then used to purify ecFcεRIα expressed in both C. reinhardtii and E. coli. As such, the column also serves to assess the activity of the receptor produced in these systems.

Functional ecFcεRIα has been expressed in the periplasm of E. coli (Robertson, 1993), and this approach is followed to generate a control molecule with which to compare C. reinhardtii produced ecFcεRIα. E. coli expressed ecFcεRIα is purified using the ecFcεRIα affinity column described above.

ELISA detection assays are carried out using standard techniques (Harlowe and Lane, 1988) to assess the affinity of E. colit expressed ecFcεRIα vs the C. reinhardtii expressed counterpart for JW8 antibody. Similarly, the affinity of functional (as judged by ELISA) ecFcεRIα-IgG1 (expressed in either the mammalian or E. coli system) for JW8, can be assessed vs. the C. reinhardtii expressed counterpart.

While ELISA assays confirm that ecFcεRIα and ecFcεRIα-IgG1 expressed in C. reinhardtii are capable of binding JW8 (hIgE), the assay does not confirm that these molecules are interacting in a manner that would specifically block hIgE interaction with ecFcεRIα displayed on the surface of a cell. In order to address this point, experiments are carried out essentially as described by Wilson et al. (1993) and Nechansky et al. (2001). Briefly, rat basophil cell line RBL-2H3 are transfected with full length FcεRIα (not simply the extracellular domain). This cell line, expressing FcεRIα, has demonstrated its ability to support IgE mediated target cell exocytosis and expression of mast cell mediators and granule components such as hexosaminidase. Transfected RBL-2H3 cells incubated with varying concentrations of JW8 are triggered with NIP-BSA as described, and levels of secreted vs total available hexosaminidase are calculated. The ability of C. reinhardtii expressed ecFcεRIα or ecFcεRIα-IgG1 to inhibit the IgE mediated release of hexosaminidase in this assay is indicative of the recombinant receptor or receptor IgG fusion's ability to interact precisely with the FcεRIα binding site on the Fc portion of hIgE. Positive controls are E. coli expressed ecFcεRIα and mammalian or E. coli expressed ecFcεRIα-IgG1.

### Example 1

### Expression of Fc-Fusion Proteins

A mammalian soluble receptor was fused to the hinge, C_{H}2 and C_{H}3 domains of a human IgG1 molecule. The exact nature of the amino acid sequence at the receptor-Fc junction is somewhat important, as many workers have observed that not all native sequences will lead to expression/accumulation of the molecule. Often times, the precise amino acid sequence in this region must be optimized to obtain a molecule which is stable and soluble, in vivo. This construct, codon optimized for expression in the C. reinhardtii chloroplast, was then cloned downstream of the strong 16sG promoter/atpA 5′ UTR (16sG/atpA) or the psbA promoter/5′ UTR (psbA) for expression in C. reinhardtii chloroplasts. 16sG/atpA driven cassettes were introduced into the p321 vector for integration in the inverted repeat region of the C. reinhardtii chloroplast genome, while psbA driven cassettes were introduced into the Chl B region in a construct which completely ablates this non-essential gene. Using particle gun mediated transformation, each of the above cassettes was independently co-transformed with plasmid p228 (which confers resistance to spectinomycin) into the chloroplast of C. reinhardtii. Selection was carried out on TAP medium containing spectinomycin. Homoplasmic lines were generated and large scale cultures were grown to assess Fc receptor fusion purification using standard protein A or protein G affinity matrices. Data are shown below for the 16sG/atpA line purified via Protein A (Protein G affinity matrices were also demonstrated to work comparably, as was purification of receptor Fc fusion when expressed from the psbA driven cassette.

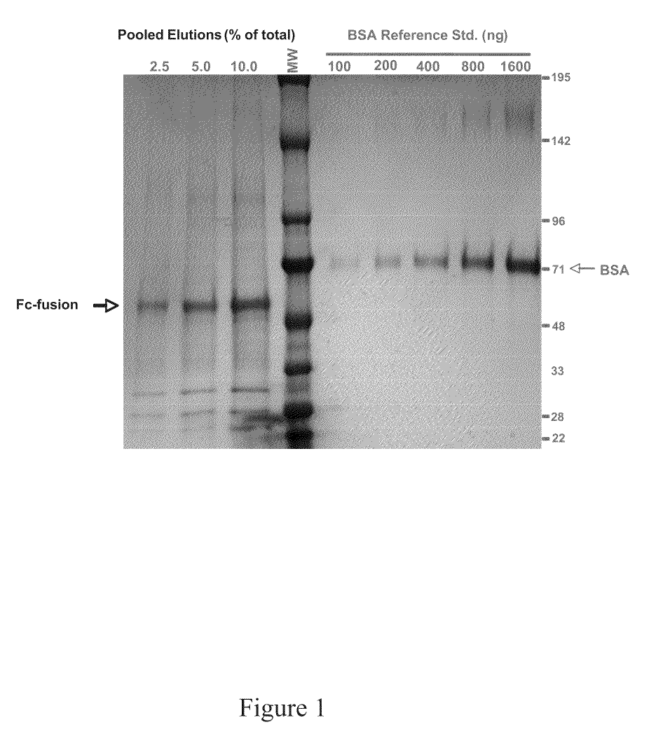

Transplastomic algae expressing the Fc-fusion protein were grown in photo-bioreactors in TAP medium in continuous batch mode. Cells were harvested via TFF followed by centrifugation and biomass was frozen at −80° C. until further use. Cells were re-suspended in lysis buffer (25 mM Tris.Cl, pH 7.4, 130 mM NaCl, 2.7 mM KCl, 1 mM PMSF, 0.1% Tween 20) with stirring. Cells were cracked at 16,000 psi in a microfluiidizer (Microfluidics, Inc.) followed by centrifugation at 4° C. and 30,000×g for 30 min. The recovered supernatant was then subjected to filtration over a 0.8 micron dead end filter (Nalgene, Inc.). Supernatant containing the Fc-fusion was then incubated with Protein A agarose for 4-12 hours with mixing. Protein A, with bound Fc-fusion protein, was subjected to column chromatography. Resin was washed with 30 column volumes of lysis buffer. Chloroplast expressed Fc-fusion protein was eluted from Protein A with 17 column volumes of 0.2M glycine.HCl, pH 2.5 into tubes containing 1M Tris.Cl, pH 9.0. Samples were then concentrated over Amicon Ultra filter devices (10,000 MW) and analyzed as shown in FIG. 1.

Data from eight independent harvests from a transgenic line expressing an Fc-receptor fusion protein is shown in Table 3.

TABLE 3
Yield of receptor-Fc fusion protein purified via chromatography
over Protein A agarose (Upstate Biotechnology, Inc.)

| | | Total Sol. | | |
|---|---|---|---|---|
| Harvest Amt (g) | Accum (% tsp) | Protein (mg) | Yield (mcg) | % Yield |
| 20 | 0.125 | 720.6 | 20 | 2.2 |
| 20 | 0.125 | 720.6 | 30 | 3.3 |
| 20 | 0.125 | 720.6 | 30 | 3.3 |
| 20 | 0.125 | 720.6 | 25 | 2.8 |
| 20 | 0.125 | 720.6 | 30 | 3.3 |
| 20 | 0.125 | 720.6 | 40 | 4.4 |
| 20 | 0.125 | 720.6 | 40 | 4.4 |
| 20 | 0.125 | 720.6 | 30 | 3.3 |

Accumulation was assessed by western blot analysis of crude lysates versus reference standard. Total soluble protein in lysate was quantified using Bradford assay (BioRad). Yield was estimated by comparing Protein A purified material to BSA standard on a coomassie stained gel. Percent yield was calculated by dividing amount of Fc-fusion captured (Yield) by (Accumulation×Total Sol. Protein).

### Example 2

### Receptor-Fc Fusions

a. IL-17 Receptor-Fc Fusions

Secreted by CD4 T cells, IL-17 is a cytokine that shows markedly elevated levels in the synovial fluid of patients with rheumatoid arthritis. IL-17 is clearly involved in the inflammatory pathway in ways similar to tumor necrosis factor (TNF). Controlling IL-17 levels through its sequestration by a soluble receptor-Fc fusion, is one approach to mitigating its pro-inflammatory properties. Possible indications where such molecules might show efficacy are RA, Crohn's disease and Irritable Bowel Syndrome.

The amino acids delimiting the extracellular domain of IL-17 receptor may be determined from the GenBank database, for example, for Gen Bank Acc. No. Q9NRM6, the extracellular domain comprises amino acids 18-292. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. The fusion protein may be purified by Protein A or Protein G affinity chromatography.

b. Receptor for TNF Alpha-Fc Fusion

Tumor necrosis factor is a critical mediator in the inflammatory response, up-regulating the expression of adhesion molecules, and promoting neutrophil and eosinophil migration to sites of inflammation. Mitigating these effects through neutralization of TNF has become a key strategy in combating diseases such as RA., both through the use of mAbs which prevent TNF interaction with its receptor and TNF a-Fc fusions, which again, act as ligand traps for TNF.

The amino acids delimiting the extracellular domain of TNF alpha receptor may be determined from the GenBank database, for example, for Gen Bank Acc. No. P20333, the extracellular domain comprises amino acids 23-257. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

c. High Affinity IgE Receptor (FcεRIα)-Fc Fusions.

The Fc portion of circulating IgE normally interacts with FcεRIα, the high affinity receptor for IgE on the surface of mast cells and basophils. By preventing FcεRIα/IgE interaction and subsequent crosslinking of receptors, the FcεRIα-Fc fusion limits the production of inflammatory mediators produced by these cells (i.e. histamine, leukotrienes, protaglandins etc.).

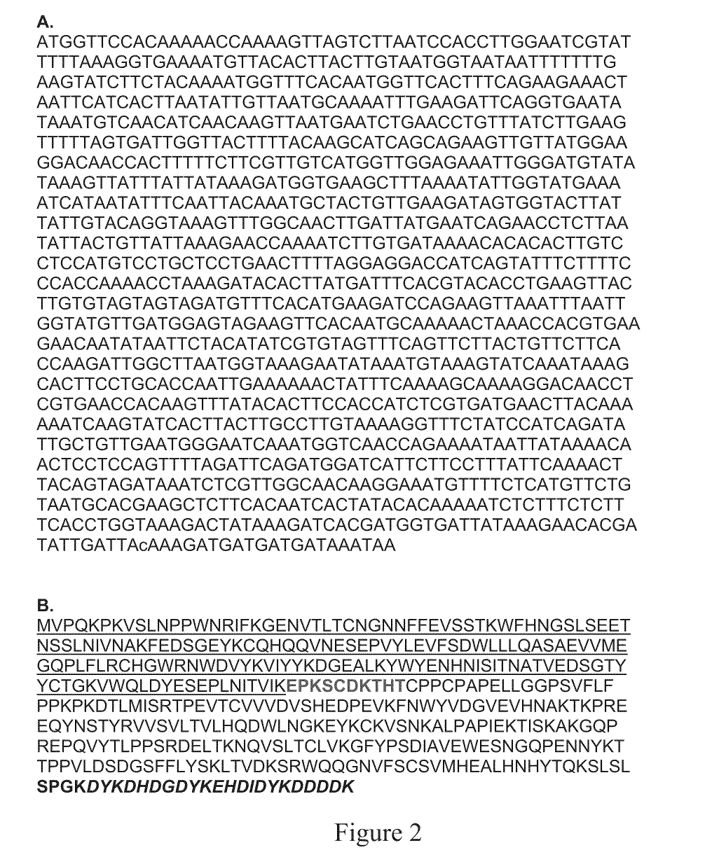

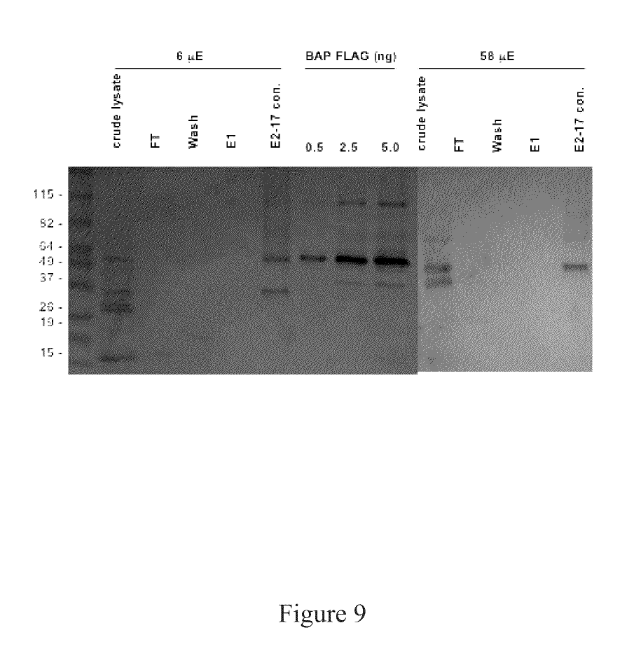

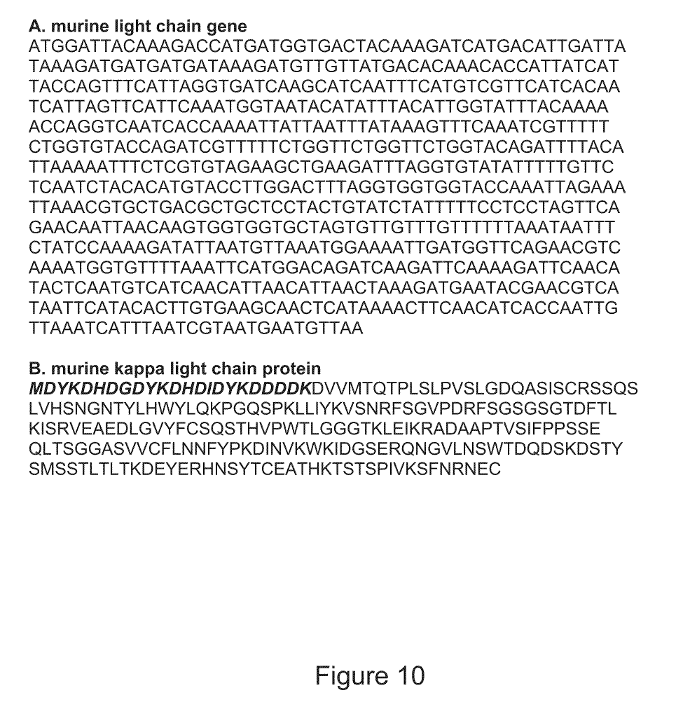

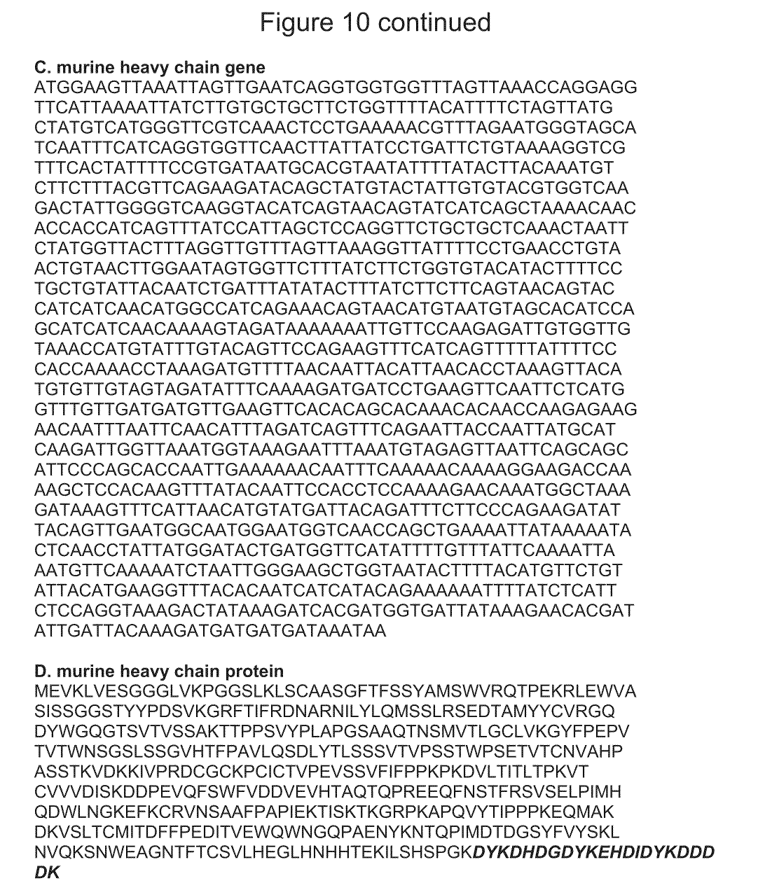

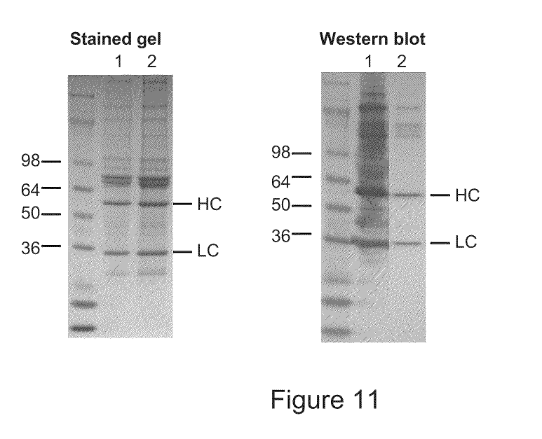

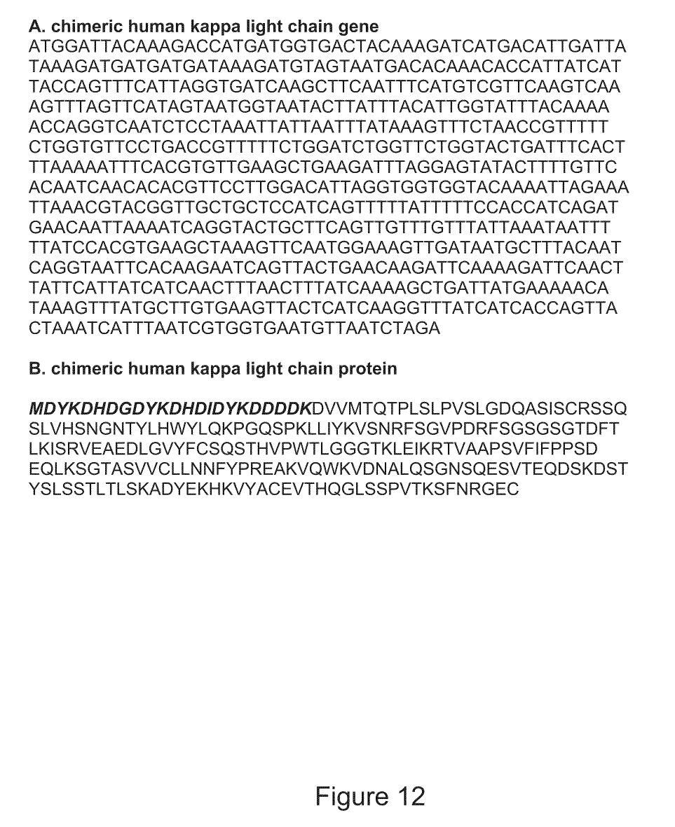

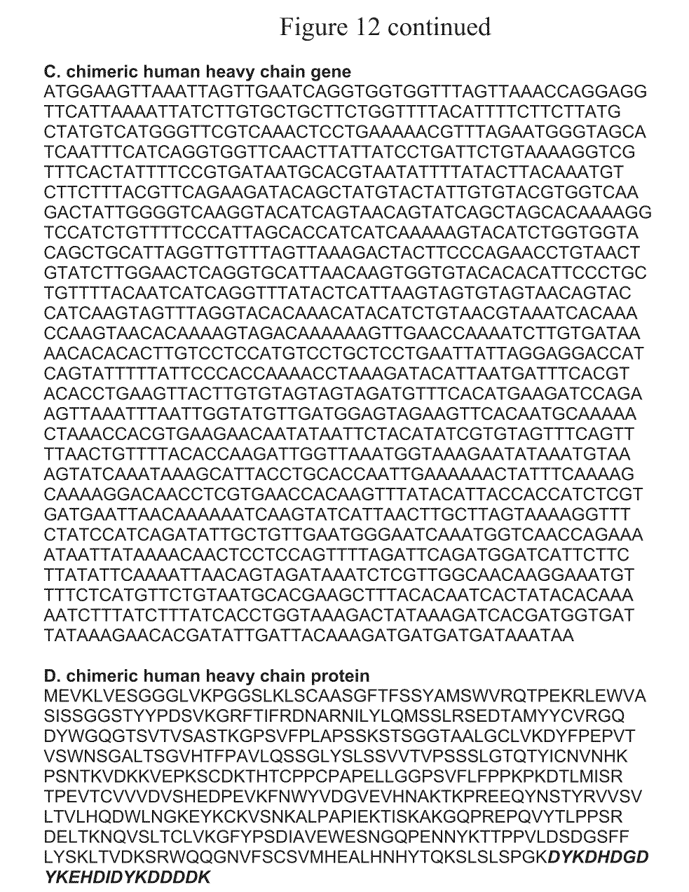

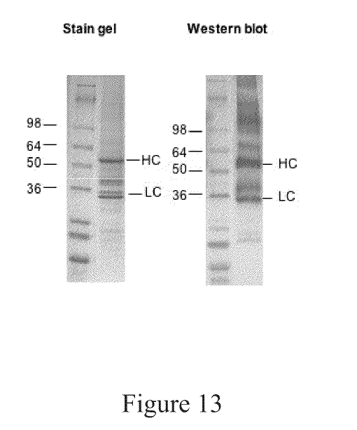

The soluble domain of the high affinity receptor for IgE antibodies was fused to the hinge, C_{H}2 and C_{H}3 domains of a human IgG1 molecule (FIG. 2). The exact nature of the amino acid sequence at the receptor-Fc junction is somewhat important, as many workers have observed that not all native sequences will lead to expression/accumulation of the molecule. Often times, the precise amino acid sequence must be optimized to obtain a molecule that is stable and soluble, in vivo. Hence, multiple forms of such a receptor-Fc fusion might be envisioned where the precise amino acid sequence at the receptor-hinge junction could be altered to improve solubility/folding characteristics of the molecule. This construct, codon optimized for expression in the C. reinhardtii chloroplast, was then cloned downstream of either the strong 16sA promoter/atpA 5′ UTR (16sA/atpA), the atpA promoter/5′UTR (atpA), the 16sA promoter/rps18 5′UTR (16sA/rps18) or the psbD promoter/5′UTR (psbD) for expression in C. reinhardtii chloroplasts. Each of these cassettes were introduced into the p321 vector for integration in the inverted repeat region of the C. reinhardtii chloroplast genome. Using particle gun mediated transformation, each of the above cassettes was independently co-transformed with plasmid p228 (which confers resistance to spectinomycin) into the chloroplast of C. reinhardtii with selection carried out on TAP/Spec medium. Homoplasmic lines were generated and large scale cultures were grown to assess our ability to purify this Fc receptor fusion using standard protein A affinity matrices. Data below (FIG. 9) are only from the psbD line, but all lines generated showed similar purification of the receptor-Fc fusion.

Frozen C. reinhardtii biomass was resuspended in TBST-pH7.4 plus 1 mM PMSF. Cells were cracked using a sonicator. Samples were subjected to centrifugation to pellet debris. The clarified lysate was incubated with Protein-A agarose (Upstate) resin overnight at 4° C. with mixing. The following day, Protein A agarose with bound alga expressed mAb was packaged into chromatography columns (BioRad Polyprep). The flow through was collected and analyzed. Columns were washed with 30 column volumes of TBST pH7.4 and the wash fractions collected for analysis. Recombinant alga mAb was eluted from Protein A using 0.2M glycine HCl, pH2.5 and the solution immediately neutralized via addition of 1M Tris.Cl, pH9.0. The antibody solution was concentrated using an Amicon Ultra 10,000 MWCO concentrator. Samples were run on 4-20% Tris Glycine gels, transferred to nitrocellulose and the membrane incubated with an murine anti-FLAG mAb followed by goat anti-mouse AP conjugated secondary polyclonal sera. Blots were developed with NBT/BCIP (FIG. 9).

d. Low Affinity IgE Receptor(FcεRII/CD23)-Fc Fusion.

Like FcεRI, FcεRII/CD23, is expressed on the surface of activated lung alveolar macrophages, monocytes and B cells of atopic individuals. Activation of the receptor on the surface of antigen presenting cells (APCs) leads to the production of pro-inflammatory cytokines, while its activation on B cells leads to increased synthesis of IgE as well as antigen presentation. Again, controlling circulating levels of IgE via such a receptor-Fc fusion has the potential to ameliorate allergic diseases including types of asthma.

The amino acids delimiting the extracellular domain of Low Affinity IgE receptor may be determined from the GenBank database, for example, for Gen Bank Acc. No. P06734, the extracellular domain comprises amino acids 48-321. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

e. A Chain High Affinity Receptor for IL-4-Fc Fusion

The cytokine Interleukin 4 is involved in isotype switching in B cells and also in the production of IgE. The a chain high affinity receptor for IL-4, fused to Fc has the potential to be used as a ligand trap for this cytokine and might find application in treating diseases such as asthma.

The amino acids delimiting the extracellular domain of a Chain High Affinity Receptor for IL-4 may be determined from the GenBank database, for example, for Gen Bank Acc. No. P24394, the extracellular domain comprises amino acids 26-232. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

f. Receptor for IL-5-Fc Fusion

Interleukin 5 promotes the recruitment, survival and activation of eosinophils. Again, such receptor-Fc fusions might find application in diseases such as asthma or hypereosinophilia syndromes.

The amino acids delimiting the extracellular domain of IL-5 receptor may be determined from the GenBank database, for example, for Gen Bank Acc. No. P24394, the extracellular domain comprises amino acids 26-232. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

g. Receptor for IL-13-Fc Fusion

Interleukin 13 has been demonstrated to play a key role in airway hyper-responsiveness, thus it is a promising target for the treatment of asthma. Fusion of the high affinity receptor for IL-13 (IL13-αR) to hIgG1 Fc domains would provide a means to block the effects of this cytokine.

The amino acids delimiting the extracellular domain of IL-13 receptor may be determined from the GenBank database, for example, for Gen Bank Ace. No. CAA70021, the extracellular domain comprises amino acids 27-343. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

h. Receptor for IL-2 (a Subunit/Cd25)-Fc Fusion

Interleukin 2 is yet another cytokine involved in the proinflammatory response cascade interaction of IL-2 with its high affinity receptor heightens T cell responses and promotes secretion of IL-4, 5 and 13. CD25-Fc fusions might find applications in allergic disease as well as suppression of T-cell responses in transplantation, for which the anti-CD25 mAb, Daclizumab, is already prescribed.

The amino acids delimiting the extracellular domain of IL-2 receptor alpha subunit may be determined from the GenBank database, for example, for Gen Bank Acc. No. NP_{—}000408, the extracellular domain comprises amino acids 22-236. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

i. Cytotoxic T Lymphocyte-Associated Antigen-4 (CTLA)-4-Fc Fusion

CD 28 on T cells interacts with both CD80 (B7-1) and CD86 (B7-2) on APCs. The interaction of CD28 with these molecules results in T cell proliferation, and cytokine release which in turn elicits macrophage and dendritic cell migration to the sites of inflammation. CTLA-4 is also expressed on T cells, and is a much higher affinity binder for CD80 and CD86. CTLA-4-Fc fusions can therefore block CD28 interactions with these molecules, preventing the inflammatory cascade described above.

The amino acids delimiting the extracellular domain of CTLA-4 may be determined from the GenBank database, for example, for Gen Bank Acc. No. NP_{—}0010032720, the extracellular domain comprises amino acids 36-161. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

j. Receptor for RANK (Receptor Activator of Nuclear Factor κβ)-Fc Fusion

RANK, a member of the TNF receptor superfamily, interacts with its cognate ligand RANKL. RANKL (also known as osteoclast differentiation factor) is responsible, through binding to RANK, for osteoclastogenesis. Blocking the RANK/RANKL interaction should limit osteoclastogenesis and hence bone resorption in general. Such an approach might have great therapeutic value in anti-resorptive therapeutic regimens, such as tumor induced hypercalcemia.

The amino acids delimiting the extracellular domain of RANK may be determined from the GenBank database, for example, for Gen Bank Acc. No. QY606, the extracellular domain comprises amino acids 30-212. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

### Example 3

### Non-Receptor-Fc Fusions

a. Factor-VII Fc Fusion (ICON)

Factor-VII is the natural ligand for the transmembrane receptor known as tissue factor. Tissue factor is selectively expressed on proliferating endothelial cells of the tumor vasculature and not in normal tissues. Age-related macular degeneration (AMD) is the cause of irreversible blindness in elderly population. In order to reduce the rate of visual loss in patients with AMD, minimizing sub-retinal choroidal neo-vascularization is of paramount importance. The Factor-VII domain in the Fc fusion binds with high affinity and specificity to tissue factor, while the aglycosylated Fc effector domain, recruits natural killer cells initiating a powerful cytolytic response against cells expressing tissue factor.

The amino acid sequence for Factor-VII is well known in the art, and may be obtained from the GenBank database, for example, Gen Bank Acc. No. AAA51983. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. The fusion protein may be purified by Protein A or Protein G affinity chromatography.

b. Glucagon-Like Protein-1 (GLP-1)-Fc Fusion
GLP-1 is a beta-cell growth factor recently identified as a promising strategy to enhance functional beta-cell mass and restore insulin secretion in patients with type 1 diabetes. Native GLP-1 has a short circulating half-life (<2 min) that results mainly from rapid enzymatic inactivation or renal clearance. Therefore, continuous subcutaneous infusion by pump is necessary to maintain GLP-1 action in vivo. GLP-1-Fc fusions have proven to have longer half-lives and are promising candidates for protecting against the onset of diabetes.

The amino acid sequence for GLP-1 is well known in the art, and may be obtained from the GenBank database, for example, Gen Bank Acc. No. NP_{—}002045. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

c. Granulocyte-Macrophage Colony Stimulating Factor (GM-CSF)-Fc Fusion

Granulocyte-Macrophage Colony Stimulating Factor (GM-CSF) is a member of the hematopoietic cytokine family, which includes interleukin-3 (IL-3) and interleukin-5 (IL-5). It is a pleiotropic cytokine that was one of the first growth factors characterized and shown to be necessary for the proliferation, differentiation, activation, and survival of hematopoietic cells. GM-CSF shows therapeutic value by accelerating neutrophil recovery in disease-induced myelo-suppression such as bone marrow transplantation, chemotherapy, and infectious disease. GM-CSF-Fc fusions are promising molecules for maintaining GM-CSF activity while increasing its circulating half-life.

The amino acid sequence for GM-CSF is well known in the art, and may be obtained from the GenBank database, for example, Gen Bank Acc. No. AAA53578. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

d. DNAse I-Fc fusion

The ability of recombinant human DNAse I to degrade DNA to lower molecular weight fragments is the basis for its therapeutic use in cystic fibrosis patients. It is inhaled into the airways where it reduces the viscoelasticity of cystic fibrosis sputum, resulting in improved lung function fewer respiratory exacerbations. DNAse I has also shown promise as an agent against systemic lupus erythematosus. DNAse I dimmers seem to have interesting properties compared to its monomers, such as an enhanced ability to degrade DNA, increased resistance to inhibition by salt etc. The fusion of DNAse I to Fc molecules would be a strategy for making such dimers and also for conferring a longer half-life on DNAse 1.

The amino acid sequence for DNAse 1 is well known in the art, and may be obtained from the GenBank database, for example, Gen Bank Acc. No. AAA63170. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

e. Extracellular Ig-Like Domain of Human Myelin Oligodendrocyte Glycoprotein (MOG)-Fc Fusion

MOG is a glycoprotein believed to be important in the process of myelinization of nerves in the central nervous system. It is a transmembrane protein expressed on the surface of oligodendrocyte cell and on the outermost surface of myelin sheaths. Interest in MOG has centered on its role in demyelinating diseases, particularly multiple sclerosis (MS). Several studies have shown a role for antibodies against MOG in the pathogenesis of MS. Aglycosylated MOG peptides have shown to retain significant activity compared to full-length MOG, but their potential short half-life makes the use of MOG-Fc fusions promising for the treatment of demyelinating diseases.

The amino acid sequence for MOG is well known in the art, and may be obtained from the GenBank database, for example, Gen Bank Acc. No. CAI95562. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

f. CD24-Fc Fusion

CD24, A glycosyl-phosphotidyl-inositol (GPI) anchored protein, has been identified as a genetic modifier for risk and progression of multiple sclerosis. It has also been shown to significantly reduce T cell activation. The extracellular domain of CD24 is a scant 31 amino acids, hence, its half life in sera would be exceedingly short. Fusion of CD24 to Fc domains would make this molecule a potentially valuable therapeutic in the treatment of MS and other autoimmune disorders.

The amino acid sequence for CD24 is well known in the art, and may be obtained from the GenBank database, for example, Gen Bank Acc. No. AAH64619. A chloroplast biased nucleotide sequence is generated which encodes extracellular domain (see Franklin et al. Plant J (2002) 30:733-744, Mayfield et al., Proc Natl Acad Sci USA (2003) 100:438-442, Mayfield et al., Plant J (2004) 37:449-458) using PCR based oligonucleotide gene assembly (Stemmer et al., Gene (1995) 164:49-53). Once assembled, the sequence will be linked the hinge, C_{H}2-C_{H}3 domains of heavy chain hIgG1 using an in frame amino acid linker (low complexity) located between the carboxy terminus of the ECD of the receptor and amino terminus of the Fc region. Again, the fusion protein may be purified by Protein A or Protein G affinity chromatography.

g. Expression and Purification of Murine IgG1 mAb Via Protein G Affinity

An extremely useful antibody, termed NT73, was developed from an immunization with core E. coli RNA polymerase (Thompson et al., 1992. Biochemistry, 1992. 31: 7003-8). It recognizes an epitope on the β′ subunit of this multisubunit enzyme and this epitope was eventually mapped to a 13 amino acid or smaller sequence at the N-terminal end of the β′ subunit (Thompson et al., 2003. Anal. Biochem. 2003, 323:171-179.). It also functions to recognize this epitope when transferred to a heterologous protein via recombinant DNA cloning. This has made the antibody a very useful reagent for immunopurification of epitope tagged proteins. We have sequenced the mRNAs encoding the murine light and heavy chain genes via RT-PCR. Genes encoding both the light and heavy chain proteins were synthesized de-novo and the codon usage of both genes was optimized for expression in the C. reinhardtii chloroplast. Light chain gene expression was driven by the strong 16sA promoter/atpA 5′ UTR (16sA/atpA) while heavy chain expression was driven by the chlB promoter/5′UTR. Light chain cassettes, containing an N-terminal FLAG Tag, were introduced into the p321 vector for integration in the inverted repeat region of the C. reinhardtii chloroplast genome, while heavy chain cassettes, containing a C-terminal FLAG Tag, were introduced as direct replacements into the Chl B region in a construct which completely ablates this non-essential gene. Using particle gun mediated transformation, both of the above cassettes were co-transformed with plasmid p228 (which confers resistance to spectinomycin) into the chloroplast of C. reinhardtii. Homoplasmic lines were generated and large scale cultures were grown to assess purification of this mIgG1 mAb using Protein G affinity matrices.

h. Murine IgG1 Purification.

Frozen C. reinhardtii biomass (53.1 g) was resuspended in TBST-pH7.4 plus 1 mM PMSF. Cells were cracked using a Micriofluidics microfluidizer operating at 16,000 psi, the debris pelleted and the lysate filtered through a 0.8 um dead end filter followed by a 0.2 um dead end filter. The lysate was incubated with Protein-G agarose (Upstate) resin overnight at 4 deg C. with mixing. The following day, Protein G agarose with bound alga expressed mAb was packaged into chromatography columns (BioRad Polyprep). the flow through was collected and analyzed. Columns were washed with 30 column volumes of TBST pH 7.4 and the wash fractions collected for analysis. Recombinant mAb was eluted from the Protein G matrix using 0.2M glycine HCl, pH 2.5 and the solution immediately neutralized via addition of 1M Tris.Cl, pH 9.0. The antibody solution was concentrated to 90 ng/ul in a final volume of 196.8 ul, using an Amicon Ultra 10,000 MWCO concentrator.

We also developed a chimeric murine-human version of the NT73 antibody molecule by cloning the murine light and heavy chain variable domains onto human light and heavy chain constant domains. In this instance, light chain and heavy chain gene expression were both driven by the strong 16sA promoter/atpA 5′ UTR (16sA/atpA). Light chain cassettes, containing an N-terminal FLAG Tag, were introduced into the p321 vector for integration in the inverted repeat region of the C. reinhardtii chloroplast genome, while heavy chain cassettes, containing a C-terminal FLAG Tag, were introduced into a p3HB vector allowing for integration in the psbH region of the chloroplast genome. This vector contains a functional copy of the psbH gene. When transformed into a strain containing a disrupted copy of this gene, transformants at this locus can be selected for their ability to grow on minimal media. Using particle gun mediated transformation, both of the above cassettes were co-transformed with plasmid p228 (which confers resistance to spectinomycin) into the chloroplast of C. reinhardtii strain 137c(+)/psbH(−) and selected for their ability to grow on minimal/spec medium. Homoplasmic lines were generated and large-scale cultures were grown to assess our ability to purify this hIgG1 mAb using standard Protein A affinity matrices.

i. Chimeric Human IgG1 Purification

Frozen. C. reinhardtii biomass (92.4 g) was treated exactly as described for the mIgG1 except that purification was effected over Protein A (Upstate). Algal-expressed hIgG1 was concentrated to 160 ng/ul in a final volume of 250 ul.

Although the invention has been described with reference to the above examples, it will be understood that modifications and variations are encompassed within the spirit and scope of the invention. Accordingly, the invention is limited only by the following claims.

What is claimed is:

1. A method for producing an aglycosylated bifunctional fusion protein comprising, transforming a plant or algal plastid with one or more expression constructs, said one or more constructs comprising in operable linkage, a nucleic acid signal sequence element for homologous recombination and expression of a fusion protein in a plastid, a first polynucleotide sequence encoding a fragment crystallizable (Fc) region or fragment thereof, and a second polynucleotide sequence encoding an extracellular domain (ECD) of a receptor or surface glycoprotein, a growth factor, or an enzyme, wherein said first and said second polynucleotides are expressed as a fusion protein; and
expressing said construct to produce an aglycosylated bifunctional fusion protein.

2. The method of claim 1, wherein said fusion protein mediates binding to an FcRN receptor, Protein A or Protein G, but not to Fc receptors the mediate opsonization, cell lysis, or degranulation of mast cells, basophils or eosinophils.

3. The method of claim 2, wherein said Fc region comprises an IgG1Fc, an IgG2Fc, an IgG3Fc, an IgG4Fc, an IgAFc, an IgEFc, an IgMFc, an IgDFc or a fragment thereof.

4. The method of claim 1, wherein said ECD of a receptor comprises a cytokine receptor ECD, an immunoglobulin receptor ECD, a T-cell receptor ECD, a cluster of differentiation antigen receptor ECD, a growth factor receptor ECD, a tissue factor receptor ECD, or a blood factor receptor ECD.

5. The method of claim 1, wherein said ECD of a receptor comprises an FcεRIα ECD (ecFcεRIα), an IL-17 receptor ECD, a TNFα receptor ECD, a high affinity IgE receptor ECD, a low affinity IgE receptor ECD, an α chain high affinity IL-4 receptor ECD, an IL-5 receptor ECD, an IL-13 receptor ECD, an IL-2 receptor ECD, a cytotoxic T-lymphocyte-associated antigen 4 (CTLA-4), a receptor activator of nuclear factor κβ (RANK) or tissue factor.

6. The method of claim 1, wherein said growth factor is TGF-β, G-CSF, GM-CSF, NGF, BDNF, NT3, PDGF, EPO, TPO, myostatin, GD9F, bFGF, EGF or HGF.

7. The method of claim 4, wherein said cytokine for said receptor ECD is IL-1α, IL-2, IL-3, IL-4, IL-5, IL-6, IL-7, IL-8, IL-9, IL-10, IL-12, IL-13, IL-14, IL-15, IL-17, INF-α, INF-β, INF-γ, MIP-1a, MIP-1b, RANTES, MCP-1, MCP-2, MCP-3, MCP-4, or PF-4.

8. The method of claim 1, wherein said surface glycoprotein is an integrin, an immunoglobulin superfamily member, a selectin, or a cadherin.

9. The method of claim 1, wherein said fusion protein modulates antibody-dependent cell mediated cytotoxicity (ADCC).

10. The method of claim 1, wherein said Fc domain is a human IgG1Fc.

11. The method of claim 10, wherein said ECD of a receptor comprises an FcεRIα ECD (ecFcεRIα).

12. The method of claim 1, further comprising purifying said fusion protein.

13. The method of claim 12, wherein said purifying utilizes Protein A or Protein G.

14. A nucleic acid construct comprising in operable linkage, nucleic acid signaling elements for homologous recombination and expression of a fusion protein in a plant or algal plastid; a first polynucleotide sequence encoding a fragment crystallizable (Fc) region; and a second polynucleotide encoding an extracellular domain (ECD) of a receptor or surface glycoprotein, a growth factor, a cytokine, or an enzyme, wherein said first and second polynucleotide sequences are expressed as a fusion protein.

15. The construct of claim 14, wherein said Fc region comprises IgG1Fc, IgG2Fc, IgG3Fc, IgG4Fc, IgAFc, IgEFc, IgMFc or IgDFc.

16. The construct of claim 14, wherein said ECD of a receptor comprises a cytokine receptor ECD, an immunoglobulin receptor ECD, a T-cell receptor ECD, a cluster of differentiation antigen receptor ECD, a growth factor receptor ECD, a tissue factor receptor ECD or a blood factor receptor ECD.

17. The construct of claim 14, wherein said ECD of a receptor comprises an FcεRIα ECD (ecFcεRIα), an IL-17 receptor ECD, a TNFα receptor ECD, a high affinity IgE receptor ECD, a low affinity IgE receptor ECD, an α chain high affinity IL-4 receptor ECD, an IL-5 receptor ECD, an IL-13 receptor ECD, an IL-2 receptor ECD, a cytotoxic T-lymphocyte-associated antigen 4 (CTLA-4), a receptor activator of nuclear factor κβ (RANK) or tissue factor.

18. The construct of claim 14, wherein said growth factor is TGF-β, G-CSF, GM-CSF, NGF, BDNF, NT3, PDGF, EPO, TPO, myostatin, GD9F, bFGF, EGF or HGF.

19. The construct of claim 16, wherein said cytokine for said receptor ECD is IL-1α, IL-1β, IL-2, IL-3, IL-4, IL-5, IL-6, IL-7, IL-8, IL-9, IL-10, IL-12, IL-13, IL-14, IL-15, IL-17, INF-α, INF-β, INF-γ, MIP-1a, MIP-1b, RANTES, MCP-1, MCP-2, MCP-3, MCP-4, or PF-4.

20. The construct of claim 14, wherein said enzyme is a DNAse I.

21. The construct of claim 14, wherein said surface glycoprotein is an integrin, a immunoglobulin superfamily member, a selectin, or a cadherin.

22. The construct of claim 21, wherein said surface glycoprotein is an extracellular Ig-like domain of human myelin oligodendrocyte glycoprotein (MOG).

23. The construct of claim 15, wherein said Fc domain is a human IgG1Fc.

24. The construct of claim 23, wherein said ECD of a receptor comprises an FcεRIα ECD (ecFcεRIα).

25. A plant cell, an algal cell or a progeny thereof comprising the construct of claim 1 stably integrated into a plastid of said cell.
